SEQUENCE LISTING (18 sequences)

SEQ ID NO: 1 - protein, 501 aa, Homo sapiens
MSALRRKFGDDYQVVTTSSSGSGLQPQGPGQDPQQQLVPKKKRQRFVDKNGRCNVQHGNLGSETSRYLSDLFTTLVDLKWRWNLFIFILTYTVAWLFMASMWWVIAYTRGDLNKAHVGNYTPCVANVYNFPSAFLFFIETEATIGYGYRYITDKCPEGIILFLFQSILGSIVDAFLIGCMFIKMSQPKKRAETLMFSEHAVISMRDGKLTLMFRVGNLRNSHMVSAQIRCKLLKSRQTPEGEFLPLDQLELDVGFSTGADQLFLVSPLTICHVIDAKSPFYDLSQRSMQTEQFEIVVILEGIVETTGMTCQARTSYTEDEVLWGHRFFPVISLEEGFFKVDYSQFHATFEVPTPPYSVKEQEEMLLMSSPLIAPAITNSKERHNSVECLDGLDDITTKLPSKLQKITGREDFPKKLLRMSSTTSEKAYSLGDLPMKLQRISSVPGNSEEKLVSKTTKMLSDPMSQSVADLPPKLQKMAGGAARMEGNLPAKLRKMNSDRFT

SEQ ID NO: 2 - protein, 501 aa, Homo sapiens
MSALRRKFGDDYQVVTTSSSGSGLQPQGPGQDPQQQLVPKKKRQRFVDKNGRCNVQHGNLGSETSRYLSDLFTTLVDLKWRWNLFIFILTYTVAWLFMASMWWVIAYTRGDLNKAHVGNYTPCVANVYNFPSAFLFSIETEATIGYGYRYITDKCPEGIILFLFQSILGSIVDAFLIGCMFIKMSQPKKRAETLMFSEHAVISMRDGKLTLMFRVGNLRNSHMVSAQIRCKLLKSRQTPEGEFLPLDQLELDVGFSTGADQLFLVSPLTICHVIDAKSPFYDLSQRSMQTEQFEIVVILEGIVETTGMTCQARTSYTEDEVLWGHRFFPVISLEEGFFKVDYSQFHATFEVPTPPYSVKEQEEMLLMSSPLIAPAITNSKERHNSVECLDGLDDITTKLPSKLQKITGREDFPKKLLRMSSTTSEKAYSLGDLPMKLQRISSVPGNSEEKLVSKTTKMLSDPMSQSVADLPPKLQKMAGGAARMEGNLPAKLRKMNSDRFT

SEQ ID NO: 3 - DNA, 1503 bp, Homo sapiens
atgagcgccctgagaagaaagttcggcgacgactaccaggtggtcaccacaagcagctctggctctggactgcagcctcaaggacctggacaggatcctcagcagcagctggtgcccaagaagaaacggcagagattcgtggacaagaacggccggtgcaatgtgcagcacggcaatctgggcagcgagacaagcagatacctgagcgacctgttcaccacactggtggacctgaagtggcggtggaacctgttcatcttcatcctgacctacaccgtggcctggctgttcatggccagcatgtggtgggttatcgcctacaccagaggcgacctgaacaaggcccatgtgggcaactacaccccttgcgtggccaacgtgtacaacttccctagcgccttcctgttcagcatcgagacagaggccacaatcggctacggctaccggtacatcaccgacaagtgccccgagggaattatcctgttcctgttccagtccatcctgggctctatcgtggatgccttcctgatcggctgcatgttcatcaagatgagccagcctaagaagcgggccgagacactgatgtttagcgagcacgccgtgatcagcatgcgcgacggaaaactgaccctgatgttcagagtgggcaacctgcggaacagccacatggtgtcagcccagatcagatgcaagctgctgaagtccagacagacccctgagggcgagttcctgcctctggatcagctggaactggacgtgggctttagcacaggcgccgatcagctgtttctggtgtcccctctgaccatctgccacgtgatcgacgctaagagccccttctacgatctgagccagcggagcatgcagaccgagcagtttgagatcgtggtcatcctggaaggcattgtggaaaccaccggcatgacctgccaggccagaaccagctacacagaggatgaggtgctgtggggccacagattcttccccgtgatctctctggaagagggcttcttcaaggtggactacagccagttccacgccacctttgaggtgcccacacctccttacagcgtgaaagaacaagaggaaatgctgctgatgagcagccctctgatcgcccctgccatcaccaacagcaaagaacggcacaacagcgtggaatgcctggacggcctggatgacatcaccaccaagctgccttccaagctgcagaagatcaccggcagagaggacttccccaagaaactgctgagaatgagcagcaccaccagcgagaaggcctactctctgggcgacctgcctatgaagctgcagcggatctctagcgtgcccggcaactctgaggaaaagctggtttccaagaccaccaagatgctgagcgatcccatgagccagtccgtggctgatctgccacctaaactgcagaaaatggctggcggagccgccagaatggaaggcaatctgcctgccaaactgcgcaagatgaactccgaccggttcacc

SEQ ID NO: 4 - protein, 501 aa, Mus musculus
MSALRRKFGDDYQVVTTSSSGSGLQPQGPGQGPQQQLVPKKKRQRFVDKNGRCNVQHGNLGSETSRYLSDLFTTLVDLKWRWNLFIFILTYTVAWLFMASMWWVIAYTRGDLNKAHVGNYTPCVANVYNFPSAFLFFIETEATIGYGYRYITDKCPEGIILFLFQSILGSIVDAFLIGCMFIKMSQPKKRAETLMFSEHAVISMRDGKLTLMFRVGNLRNSHMVSAQIRCKLLKSRQTPEGEFLPLDQLELDVGFSTGADQLFLVSPLTICHVIDAKSPFYDLSQRSMQTEQFEVVVILEGIVETTGMTCQARTSYTEDEVLWGHRFFPVISLEEGFFKVDYSQFHATFEVPTPPYSVKEQEEMLLMSSPLIAPAITNSKERHNSVECLDGLDDISTKLPSKLQKITGREDFPKKLLRMSSTTSEKAYSLGDLPMKLQRISSVPGNSEEKLVSKTTKMLSDPMSQSVADLPPKLQKMAGGPTRMEGNLPAKLRKMNSDRFT

SEQ ID NO: 5 - protein, 501 aa, Mus musculus
MSALRRKFGDDYQVVTTSSSGSGLQPQGPGQGPQQQLVPKKKRQRFVDKNGRCNVQHGNLGSETSRYLSDLFTTLVDLKWRWNLFIFILTYTVAWLFMASMWWVIAYTRGDLNKAHVGNYTPCVANVYNFPSAFLFSIETEATIGYGYRYITDKCPEGIILFLFQSILGSIVDAFLIGCMFIKMSQPKKRAETLMFSEHAVISMRDGKLTLMFRVGNLRNSHMVSAQIRCKLLKSRQTPEGEFLPLDQLELDVGFSTGADQLFLVSPLTICHVIDAKSPFYDLSQRSMQTEQFEVVVILEGIVETTGMTCQARTSYTEDEVLWGHRFFPVISLEEGFFKVDYSQFHATFEVPTPPYSVKEQEEMLLMSSPLIAPAITNSKERHNSVECLDGLDDISTKLPSKLQKITGREDFPKKLLRMSSTTSEKAYSLGDLPMKLQRISSVPGNSEEKLVSKTTKMLSDPMSQSVADLPPKLQKMAGGPTRMEGNLPAKLRKMNSDRFT

SEQ ID NO: 6 - protein, 410 aa, Homo sapiens
MVLKAEHTRSPSATLPSNVPSCRSLSSSEDGPSGPSSLADGGLAHNLQDSVRHRILYLSEQLRVEKASRDGNTVSYLKLVSKADRHQVPHIQQAFEKVNQRASATIAQIEHRLHQCHQQLQELEEGCRPEGLLLMAESDPANCEPPSEKALLSEPPEPGGEDGPVNLPHASRPFILESRFQSLQQGTCLETEDVAQQQNLLLQKVKAELEEAKRFHISLQESYHSLKERSLTDLQLLLESLQEEKCRQALMEEQVNGRLQGQLNEIYNLKHNLACSEERMAYLSYERAKEIWEITETFKSRISKLEMLQQVTQLEAAEHLQSRPPQMLFKFLSPRLSLATVLLVFVSTLCACPSSLISSRLCTCTMLMLIGLGVLAWQRWRAIPATDWQEWVPSRCRLYSKDSGPPADGP

SEQ ID NO: 7 - protein, 7 aa, Artificial Sequence
  Synthetic insertion peptide
LGETTRP

SEQ ID NO: 8 - protein, 732 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTR

SEQ ID NO: 9 - protein, 735 aa, adeno-associated virus 2 [sequence omitted]

SEQ ID NO: 10 - protein, 10 aa, Artificial Sequence
  Synthetic insertion peptide
AALGETTRPA

SEQ ID NO: 11 - protein, 9 aa, Artificial Sequence
  Synthetic insertion peptide
LALGETTRP

SEQ ID NO: 12 - protein, 9 aa, Artificial Sequence
  Synthetic insertion peptide
GLGETTRPA

SEQ ID NO: 13 - DNA, 1724 bp, Artificial Sequence
  Synthetic pR1.7 promoter
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 14 - DNA, 234 bp, Artificial Sequence
  Synthetic minimal M-opsin promoter region
ccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccag

SEQ ID NO: 15 - DNA, 1958 bp, Artificial Sequence
  Synthetic pMNTC expression cassette
cctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggcccagagaggagacaggccgccacc

SEQ ID NO: 16 - DNA, 6493 bp, Artificial Sequence
  Synthetic plasmid
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccactagtgtaagtttagtctttttgtcttttatttcaggtcccggatccggtggtggtgcaaatcaaagaactgctcctcagtggatgttgcctttacttctagcaagtttgtacaaaaaagcaggctgccaccatgagcgccctgagaagaaagttcggcgacgactaccaggtggtcaccacaagcagctctggctctggactgcagcctcaaggacctggacaggatcctcagcagcagctggtgcccaagaagaaacggcagagattcgtggacaagaacggccggtgcaatgtgcagcacggcaatctgggcagcgagacaagcagatacctgagcgacctgttcaccacactggtggacctgaagtggcggtggaacctgttcatcttcatcctgacctacaccgtggcctggctgttcatggccagcatgtggtgggttatcgcctacaccagaggcgacctgaacaaggcccatgtgggcaactacaccccttgcgtggccaacgtgtacaacttccctagcgccttcctgttcagcatcgagacagaggccacaatcggctacggctaccggtacatcaccgacaagtgccccgagggaattatcctgttcctgttccagtccatcctgggctctatcgtggatgccttcctgatcggctgcatgttcatcaagatgagccagcctaagaagcgggccgagacactgatgtttagcgagcacgccgtgatcagcatgcgcgacggaaaactgaccctgatgttcagagtgggcaacctgcggaacagccacatggtgtcagcccagatcagatgcaagctgctgaagtccagacagacccctgagggcgagttcctgcctctggatcagctggaactggacgtgggctttagcacaggcgccgatcagctgtttctggtgtcccctctgaccatctgccacgtgatcgacgctaagagccccttctacgatctgagccagcggagcatgcagaccgagcagtttgagatcgtggtcatcctggaaggcattgtggaaaccaccggcatgacctgccaggccagaaccagctacacagaggatgaggtgctgtggggccacagattcttccccgtgatctctctggaagagggcttcttcaaggtggactacagccagttccacgccacctttgaggtgcccacacctccttacagcgtgaaagaacaagaggaaatgctgctgatgagcagccctctgatcgcccctgccatcaccaacagcaaagaacggcacaacagcgtggaatgcctggacggcctggatgacatcaccaccaagctgccttccaagctgcagaagatcaccggcagagaggacttccccaagaaactgctgagaatgagcagcaccaccagcgagaaggcctactctctgggcgacctgcctatgaagctgcagcggatctctagcgtgcccggcaactctgaggaaaagctggtttccaagaccaccaagatgctgagcgatcccatgagccagtccgtggctgatctgccacctaaactgcagaaaatggctggcggagccgccagaatggaaggcaatctgcctgccaaactgcgcaagatgaactccgaccggttcacctagacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 7084 bp, Artificial Sequence
  Synthetic plasmid
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccactagtgtaagtttagtctttttgtcttttatttcaggtcccggatccggtggtggtgcaaatcaaagaactgctcctcagtggatgttgcctttacttctagcaagtttgtacaaaaaagcaggctgccaccatgagcgccctgagaagaaagttcggcgacgactaccaggtggtcaccacaagcagctctggctctggactgcagcctcaaggacctggacaggatcctcagcagcagctggtgcccaagaagaaacggcagagattcgtggacaagaacggccggtgcaatgtgcagcacggcaatctgggcagcgagacaagcagatacctgagcgacctgttcaccacactggtggacctgaagtggcggtggaacctgttcatcttcatcctgacctacaccgtggcctggctgttcatggccagcatgtggtgggttatcgcctacaccagaggcgacctgaacaaggcccatgtgggcaactacaccccttgcgtggccaacgtgtacaacttccctagcgccttcctgttcagcatcgagacagaggccacaatcggctacggctaccggtacatcaccgacaagtgccccgagggaattatcctgttcctgttccagtccatcctgggctctatcgtggatgccttcctgatcggctgcatgttcatcaagatgagccagcctaagaagcgggccgagacactgatgtttagcgagcacgccgtgatcagcatgcgcgacggaaaactgaccctgatgttcagagtgggcaacctgcggaacagccacatggtgtcagcccagatcagatgcaagctgctgaagtccagacagacccctgagggcgagttcctgcctctggatcagctggaactggacgtgggctttagcacaggcgccgatcagctgtttctggtgtcccctctgaccatctgccacgtgatcgacgctaagagccccttctacgatctgagccagcggagcatgcagaccgagcagtttgagatcgtggtcatcctggaaggcattgtggaaaccaccggcatgacctgccaggccagaaccagctacacagaggatgaggtgctgtggggccacagattcttccccgtgatctctctggaagagggcttcttcaaggtggactacagccagttccacgccacctttgaggtgcccacacctccttacagcgtgaaagaacaagaggaaatgctgctgatgagcagccctctgatcgcccctgccatcaccaacagcaaagaacggcacaacagcgtggaatgcctggacggcctggatgacatcaccaccaagctgccttccaagctgcagaagatcaccggcagagaggacttccccaagaaactgctgagaatgagcagcaccaccagcgagaaggcctactctctgggcgacctgcctatgaagctgcagcggatctctagcgtgcccggcaactctgaggaaaagctggtttccaagaccaccaagatgctgagcgatcccatgagccagtccgtggctgatctgccacctaaactgcagaaaatggctggcggagccgccagaatggaaggcaatctgcctgccaaactgcgcaagatgaactccgaccggttcacctagacccagctttcttgtacaaagtgggaattcatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttatcatcgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 7095 bp, Artificial Sequence
  Synthetic plasmid
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccactagtgtaagtttagtctttttgtcttttatttcaggtcccggatccggtggtggtgcaaatcaaagaactgctcctcagtggatgttgcctttacttctagcaagtttgtacaaaaaagcaggctgccaccatgagcgccctgagaagaaagttcggcgacgactaccaggtggtcaccacaagcagctctggctctggactgcagcctcaaggacctggacaggatcctcagcagcagctggtgcccaagaagaaacggcagagattcgtggacaagaacggccggtgcaatgtgcagcacggcaatctgggcagcgagacaagcagatacctgagcgacctgttcaccacactggtggacctgaagtggcggtggaacctgttcatcttcatcctgacctacaccgtggcctggctgttcatggccagcatgtggtgggttatcgcctacaccagaggcgacctgaacaaggcccatgtgggcaactacaccccttgcgtggccaacgtgtacaacttccctagcgccttcctgttcagcatcgagacagaggccacaatcggctacggctaccggtacatcaccgacaagtgccccgagggaattatcctgttcctgttccagtccatcctgggctctatcgtggatgccttcctgatcggctgcatgttcatcaagatgagccagcctaagaagcgggccgagacactgatgtttagcgagcacgccgtgatcagcatgcgcgacggaaaactgaccctgatgttcagagtgggcaacctgcggaacagccacatggtgtcagcccagatcagatgcaagctgctgaagtccagacagacccctgagggcgagttcctgcctctggatcagctggaactggacgtgggctttagcacaggcgccgatcagctgtttctggtgtcccctctgaccatctgccacgtgatcgacgctaagagccccttctacgatctgagccagcggagcatgcagaccgagcagtttgagatcgtggtcatcctggaaggcattgtggaaaccaccggcatgacctgccaggccagaaccagctacacagaggatgaggtgctgtggggccacagattcttccccgtgatctctctggaagagggcttcttcaaggtggactacagccagttccacgccacctttgaggtgcccacacctccttacagcgtgaaagaacaagaggaaatgctgctgatgagcagccctctgatcgcccctgccatcaccaacagcaaagaacggcacaacagcgtggaatgcctggacggcctggatgacatcaccaccaagctgccttccaagctgcagaagatcaccggcagagaggacttccccaagaaactgctgagaatgagcagcaccaccagcgagaaggcctactctctgggcgacctgcctatgaagctgcagcggatctctagcgtgcccggcaactctgaggaaaagctggtttccaagaccaccaagatgctgagcgatcccatgagccagtccgtggctgatctgccacctaaactgcagaaaatggctggcggagccgccagaatggaaggcaatctgcctgccaaactgcgcaagatgaactccgaccggttcacctagacccagctttcttgtacaaagtgggaattcgataatcaacctctggattacaaaatttgtgaaagattgactggtattcttaactttgttgctccttttacgctttgtggatacgctgctttattgcctttgtatcttgctattgcttcccgtttggctttcattttctcctccttgtataaatcctggttgctgtctctttttgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2026/0048089 A1
(43) Pub. Date: Feb. 19, 2026

(54) G-PROTEIN-GATED-K+ CHANNEL-MEDIATED ENHANCEMENTS IN LIGHT SENSITIVITY IN ROD-CONE DYSTROPHY (RCD)

(71) Applicants: SPARINGVISION, PARIS (FR); RUHR-UNIVERSITAT BOCHUM, BOCHUM (DE); Institut National de la Santé et de la Recherche Médicale, PARIS (FR); CENTRE NATIONAL DE LA RECHERCHE SCIENTIFIQUE - CNRS -, PARIS (FR); SORBONNE UNIVERSITE, PARIS (FR)

(72) Inventors: Deniz DALKARA, Paris (FR); Stephan HERLITZE, BOCHUM (DE); Brix MÜCHER, BOCHUM (DE); Hanen KHABOU, NOISY LE GRAND (FR)

(21) Appl. No.: 18/563,959

(22) PCT Filed: May 25, 2022
(86) PCT No.: PCT/EP2022/064349

§ 371 Date: Nov. 24, 2023

(30) Foreign Application Priority Data
May 26, 2021 (EP) 21305686.4

(51) Int. Cl. A61K 35/76; A61K 38/17; A61K 48/00; A61P 27/02; C07K 14/705; C07K 14/72; C12N 15/86

(52) U.S. Cl. CPC A61K 35/76; A61K 48/0058; A61K 48/0075; A61P 27/02; C07K 14/705; C07K 14/723; C12N 15/86; A61K 38/177

(57) ABSTRACT

The present invention concerns a new gene therapy approach to increase light-sensitivity in degenerating cones in advanced stages of rod-cone dystrophy (RCD) mediated by G-protein-gated-K+ channel (GIRK), in particular GIRK1 F137S, activated by G proteins recruited by cone opsin expressed in degenerating cones.

### SEQUENCE LISTING

This application includes as the Sequence Listing the complete contents of the accompanying text file “13500206US_sub_sequence_listing”, created Sep. 16, 2025, containing 69,816 bytes, hereby incorporated by reference.

### DESCRIPTION

### Technical Field of the Invention

The present invention concerns a new gene therapy approach to increase light-sensitivity in degenerating cones in advanced stages of rod-cone dystrophy (RCD) mediated by G-protein-gated-K+ channel (GIRK), in particular GIRK1 F137S, activated by G proteins recruited by cone opsin expressed in degenerating cones.

In the description below, references in square brackets ([ ]) refer to the list of references at the end of the text.

### State of the Art

Retina is the light sensitive tissue of the eye composed of three layers of neurons interconnected by synapses. The primary neurons of the retina are the light-sensing photoreceptors (PR), which are of two types: the rods for night vision and the cones for daylight vision. Cone-mediated vision is mostly supported by the fovea and is responsible for high acuity central vision most valuable to our daily visual tasks (Sinha et al., 2017) [1]. The light sensitive G protein coupled receptors that link photon capture to intracellular signaling leading to membrane hyperpolarization in photoreceptors are called opsins (Yau and Hardie, 2009) [2]. There is one type of rod opsin found in rods and three types of cone opsins-responsible for trichromatic vision—in the primate retina. The structural properties and phototransduction cascades are similar between these opsins.

The phototransduction cascade is composed of several proteins that are concentrated in the photoreceptor outer-segments in normal retinas (FIG. 1A). The role of the photoreceptor is to sense light via this phototransduction cascade and induce an electrical signal that is then processed and transmitted towards downstream neurons (Ebrey and Koutalos, 2001) [3].

The absorption of a photon activates the opsin composed of two parts: the protein part, and the light absorbing part, which is the retinal—a derivative of vitamin A. The latter isomerizes from 11-cis-retinal (dark adapted state) into all-trans-retinal configuration (light adapted state). As a result, the opsin becomes catalytically active recruiting the G protein transducin. The α-subunit of transducin is activated by the replacement of GDP by GTP. After, the α-subunit dissociates from the βγ-subunits to activate the membrane-associated phosphodiesterase 6 (PDE) by binding its two inhibitory y subunits. The activated PDE hydrolyses cGMP into GMP. The reduction of cGMP clones the nucleotide-gated channels (CNG) and this stops cation entry, resulting in PR hyperpolarization and reduction in glutamate release by the photoreceptor (Larhammar et al., 2009) [4].

In order to respond to another photon, this phototransduction cascade is deactivated by two mechanisms: (i) the transducin inactivates itself by hydrolyzing the bound GTP and (ii) the rhodopsin kinase (GRK) phosphorylates the opsin that interacts with the regulatory protein arrestin, leading to opsin inactivation. Retinal is then recycled by the retinal pigment epithelium (RPE) and Müller glial cells. Each and every protein of this cascade plays an important role in converting the light signal into an electrical signal conveyed to the second and third order neurons (Maeda et al., 2003) [5].

Inherited retinal degenerations are mostly due to mutations in photoreceptor or RPE cells leading to the degeneration of the rods, followed by cone outer segment degeneration eventually leading to blindness (Buch et al., 2004) [6]. Among them, rod-cone dystrophy (RCD) represents the largest category where genetic causes are highly heterogeneous. More than 60 different genes expressed in rod photoreceptors or the retinal pigment epithelium are involved (Wright et al., 2010) [7]. The first gene that has been linked to RCD is the rhodopsin gene RHO that counts for 25% of RCD autosomal dominant cases. Many other causative genes have also been identified: the cGMP-PDE subunit gene and the cyclic GMP gated channel protein a subunit gene. Although there are many causative genes, the resulting RCD phenotype is the same across different mutations (Ferrari et al., 2011) [8]. The common RCD phenotype is characterized by the progressive rod degeneration, causing night blindness, and followed by progressive peripheral cone degeneration, causing “tunnel vision”, mediated entirely by the remaining foveal cones then eventually resulting in complete blindness in the latest stages of disease. Usually, when patients are diagnosed with RCD, they already show night blindness, meaning their rods have degenerated. However, the cones remain until the late stages of the disease; particularly in the foveal region responsible for high acuity leading to tunnel vision in early stages (Li et al., 1995) [9]. In later stages of the disease, these cones lose their outer segment structures leading to complete blindness before the complete loss of the cone soma and pedicle (Li et al., 1995) [9].

In order to preserve the vision in these patients presenting light sensitive cone cell bodies, one innovative strategy is retinal gene therapy, which broadly refers to the transfer of a therapeutic gene into retinal cells to mediate a therapeutic effect (Bennet, 2017) [10]. Although the first successful clinical trials of gene therapy have focused on gene replacement, where a gene carrying a recessive mutation is replaced by a functional cDNA copy, this strategy is limited because it cannot be used for the majority of retinal degenerations (Bennet, 2017) [10]. For example, in RCD, the huge variability of mutations makes it difficult to apply to each specific mutation. Moreover, dominant mutations cannot be treated using this approach. Furthermore, in 50% of cases, the causative mutation is not elucidated or the rod photoreceptors bearing the most frequent mutations are already lost (Dalkara et al., 2015) [11]. For these reasons, mutation-independent gene therapies that can be applied beyond the loss of rods must be developed to treat a large number of patients without knowledge of the mutant gene.

Taking this goal into account, previous studies used the ectopic expression of microbial opsins, like channelrhodopsin in bipolar cells or halorhodopsin in cones PRs, to modulate membrane potential and induce a depolarization or hyperpolarization respectively (Busskamp et al., 2012; Dalkara and Sahel, 2014; Scholl et al., 2016) [12-14]. Thus, in RCD, optogenetics can be used as a therapeutic strategy to restore vision in blind retinas (Baker and Flannery, 2008) [15]. There are many parameters to consider: (i) the choice of cell target to make the most out of the retinal circuitry sending the most interpretable signal to the brain (ii) the choice of the appropriate optogenetic tool to obtain a close to natural electrophysiological response in these target cells. Concerning the second point, the relatively weak capacity of microbial opsins has been a major limitation: potential immunogenicity of using an opsin from prokaryotic species, high intensities required due to the lack of signal amplification by these directly light gated channels and pumps originating from single cell microorganisms, as in the case of halorhodopsin (Baker and Flannery, 2008) [15], (Cehajic-Kapetanovic et al., 2015; Gaub et al., 2015; Van Gelder and Kaur, 2015; van Wyk et al., 2015) [16-19]. Unlike rhodopsins or cone opsins, microbial opsins are not able to activate G protein coupled cascades such as the phototransduction cascade present in healthy retinas. One of the possible ways to go beyond the limits of microbial opsins is the use of animal opsins, which are all G protein coupled receptors. However all work in this field has so far been focused on inner retinal neurons (Berry et al., 2019; Cehajic-Kapetanovic et al., 2015; De Silva et al., 2017; Gaub et al., 2015; Lin et al., 2008; van Wyk et al., 2015) [20, 16, 21, 17, 22, 19].

In a previous study, it has been shown that light-activation of two animal cone opsins can stimulate the Gro signalling pathways in human kidney cells and in neuronal cells in vitro and in vivo (Masseck et al., 2014) [23]. This pathway is involved in fast dampening of neuronal activity and inhibition of intrinsic ion channels. However, it has also been shown that animal cone opsins can directly modulate the G protein-gated inwardly rectifying potassium channels (GIRK) upon co-expression in any given cell (Berry et al., 2019; Masseck et al., 2014) [20, 23]. GIRK channels are composed of two subunits. There are four types of subunits: GIRK1 to 4. GIRK1 and 3 cannot form homotetramers; they have to be associated with GIRK2 to be functional (Mark and Herlitze, 2000) [24]. Conversely, GIRK2 alone can form homotetramers. A single point mutant GIRK1 F137S was suggested to form functional homomeric channels (Chan et al., 1996) [31]. The GIRK channel is predominantly closed at resting membrane potentials. After its activation by the By subunit of a Gio protein, potassium ions flow out of the cell, thus, hyperpolarizing the neuron (FIG. 1B). It has therefore been possible to use vertebrate cone opsins SWO (for short wavelength opsin) and LWO (for long wavelength opsin) for repetitive Gio activation of a specific wavelength in vivo in the anxiety circuitry, and the combination of cone opsins with GIRK has proven more efficient that microbial opsins at low light intensities (Masseck et al., 2014) [23].

### DESCRIPTION OF THE INVENTION

The Inventors have thus investigated if it was possible to implement this cone opsin based system in a vision restoration setting and investigated patient retinas in preparation for clinical candidate selection. Therefore, in order to develop a light-sensitive cone reactivation strategy, the expression of the phototransduction cascade elements in cones during degeneration was first examined in two RCD mouse models. After exploring the phosphotransduction cascade in two RCD mouse models, a target molecule approach acting via Gio proteins recruited by the activation of remaining cone opsin was proposed for the first time. It has thereby created a new “short phototransduction cascade” that is independent from the expression of PDE and transducin.

First, the state of the endogeneous phototransduction cascade in degenerating cones through the progression of disease was investigated. In both mouse models, only opsin and arrestin were found to migrate to the cone cell bodies after outer segment degeneration. Thus it was hypothesized that cone reactivation based on cone opsin signaling may be feasible, which in turn will allow to recover high sensitivity vision. It was found that endogenous cone opsins were still expressed at the level of the cone cell body in rd10 mouse model (FIG. 3) suggesting the possibility of linking their activity to GIRK channels, such as GIRK2 channel, even in absence of transducin and phosphodiesterase (FIG. 1B). In this configuration, the opening of GIRK channel will allow the exit of potassium ions due to the resting membrane potential of dormant cones (Busskamp, 2010) [25]. K⁺ efflux via the GIRK channel will hyperpolarize the cones in response to light as it was seen in the two mouse models of RCD.

Next, GIRK2 channel activated by G proteins recruited by cone opsin was expressed in degenerating cones. Moreover, since the remaining opsin in the cone cell bodies is still functional and sufficient to induce a light response in the degenerated cones, the insertion of GIRK2 in all cones leads to light responses following the spectral properties of each of the opsins preserving color vision. The results pointed towards enhanced light-sensitivity in cones of RCD retinas during and after degeneration of cone outer-segments. The results thus demonstrated that vertebrate light sensitive proteins combined with GIRK channel, in particular GIRK2 channel, activated by an endogenous G protein, could improve the visual function in the two mouse models, as demonstrated by electroretinography and behaviour. This is the first time that a light insensitive mammalian ion channel has been linked to intrinsic opsins providing new avenues in vision restoration that can be implemented to increase light sensitivity even before complete outer segment degeneration. Since this new system makes use of intrinsic opsins expressed in degenerating cones, it also enables for the first time, color vision restoration/maintenance.

Similarly to the RCD mouse models, the cone opsin and the cone arrestin remain in the cone cell bodies of RP human patients (FIG. 12). This result demonstrates the feasibility of reactivating the cone function in the foveal region of RCD human patients with the short GIRK2/opsin phototransduction cascade. The activation of the remaining cone opsin by a light stimulus will trigger the short phototransduction cascade and lead to vision restoration in RCD patients, even at an intermediate or advanced stage of the disease.

This new approach has thus the potential to maintain and/or restore, high acuity and color vision requiring only low light intensities in human patients.

A clear advantage of microbial opsins is their robustness and millisecond scale kinetics (Packer et al., 2013) [26]. For systems using other opsins, it should be considered that in order to respond to another light stimulus, the cascade has to be deactivated to recover light sensitivity. In absence of this, cones may stay hyperpolarized after GIRK2 channel activation limiting their ability to modulate synaptic transmission at a movie rate compatible with motion vision. In the present approach, depolarization of the cones was made possible thanks to the arrestin that is still maintained at very late stages of the disease in both RCD models. This was noticeable in the flicker ERG traces showing responses of the retina during repetitive light stimuli and also by the improved optokinetic reflex of treated mice.

Lastly, the fact that incorporation of GIRK2 enhances existing light responses in cones even prior to complete outer segment loss offers the possibility of implementing this gene therapy in mid stages of the disease. Retinal degeneration in mice is much faster than in humans, thus a few days of therapeutic efficiency in mice is equal to several years in humans. Nonetheless, even when endogenous light responses disappear, retinas expressing GIRK2 are still able to respond to light, generating response amplitudes that correspond to remaining cone numbers. This suggests that patients with remaining foveal cones can benefit from this treatment even if they have no detectable light perception at the beginning of treatment (FIG. 10). Thus this approach can be used as long as cone cells remain. Indeed a decrease in the response of treated cones to light stimuli was recorded, which was consistent with decrease in cone numbers and the fact that we did not transduce all cones due to subretinal injection further limited the beneficial effect. AAV vectors showing better lateral spread can be used to increase transduced cone numbers beyond the bleb (Khabou et al., 2018; International patent application WO 2018134168) [27, 28]. In order to increase the therapeutic window, neurotrophic factors can be implemented alongside the approach of the present invention. Indeed, AAV-mediated secretion of neurotrophic factors such as the rod-derived cone viability factors (RdCVF), have been shown to delay cone cell death and may be combined with GIRK2 mediated sensitization (Byrne et al., 2015) [29].

It has now been found that a mutated form of GIRK1 channel, GIRK1 F137S, induces significantly more ion efflux than GIRK2 in the context of a short GIRK/opsin phototransduction cascade. This result suggests that incorporation of GIRK1 F137S in cones will provide for an improved gene therapy over GIRK2 gene therapy of RCD.

An object of the present invention is therefore a vector comprising a nucleotide sequence encoding subunit 1 of G-protein-gated inwardly rectifying potassium channel (GIRK1) F137S.

The nucleotide sequence encoding GIRK1 F137Scan be under the control; of a cone-specific promoter such as pR1.7 or a functional variant thereof, or minimal M-opsin promoter, in particular in a pMNTC expression cassette, or a GRK promoter or truncated version thereof (G Protein-Coupled Receptor Kinase, GRK1 in particular).

The vector of the present invention can further comprise a nucleotide sequence encoding a mammalian cone opsin. For example, the mammalian cone opsin is a short wavelength cone opsin (SWO), e.g. from Mus musculus or human cone opsin. Where present in the same vector, the nucleotide sequence encoding GIRK1 F137S, and the nucleotide sequence encoding a mammalian cone opsin are preferably under the control of a same promoter, in particular a cone-specific promoter such as pR1.7 or a functional variant thereof, or minimal M-opsin promoter, in particular in a pMNTC expression cassette, or a GRK promoter or truncated version thereof (G Protein-Coupled Receptor Kinase, GRK1 in particular).

For the purposes of the present invention, “GIRK1 F137S” means a nucleotide sequence encoding a mutated form of wild-type human GIRK1 (SEQ ID NO: 1) or mouse GIRK1 (SEQ ID NO: 4) comprising a substitution of Phe137 by Ser and which retain the ability to respond to light when co-expressed with an opsin. The GIRK1 F137S may differ from wild-type GIRK1 by the substitution of Phe137 by Ser, only (e.g. as in SEQ ID NO: 2), or by a limited number of mutation(s), e.g. substitution and/or deletion and/or insertion of at most 1, 2, 3, 4, or 5 of an amino acid(s), in addition to the substitution of Phe137 by Ser. For example, a nucleotide sequence encoding GIRK1 F137S comprises or consists of a nucleotide sequence encoding the polypeptide of sequence SEQ ID NO:2, or comprises or consists of the nucleotide sequence SEQ ID NO: 3, or comprises or consists of a nucleotide sequence encoding the polypeptide of sequence SEQ ID NO: 5.

Human wild-type GIRK1

| | |
|---|---|
| Amino | MSALRRKFGDDYQVVTTSSSGSGLQPQGPGQDPQQQLVPKKKRQRFVDKN |
| acid | GRCNVQHGNLGSETSRYLSDLFTTLVDLKWRWNLFIFILTYTVAWLFMAS |
| sequence | MWWVIAYTRGDLNKAHVGNYTPCVANVYNFPSAFLFF₁₃₇IETEATIGYGY |
| (501 Aa) | RYITDKCPEGIILFLFQSILGSIVDAFLIGCMFIKMSQPKKRAETLMFSE |
| | HAVISMRDGKLTLMFRVGNLRNSHMVSAQIRCKLLKSRQTPEGEFLPLDQ |
| | LELDVGFSTGADQLFLVSPLTICHVIDAKSPFYDLSQRSMQTEQFEIVVI |
| | LEGIVETTGMTCQARTSYTEDEVLWGHRFFPVISLEEGFFKVDYSQFHAT |
| | FEVPTPPYSVKEQEEMLLMSSPLIAPAITNSKERHNSVECLDGLDDITTK |
| | LPSKLQKITGREDFPKKLLRMSSTTSEKAYSLGDLPMKLQRISSVPGNSE |
| | EKLVSKTTKMLSDPMSQSVADLPPKLQKMAGGAARMEGNLPAKLRKMNSD |
| | RFT |
| | (SEQ ID NO: 1) |

Human GIRK1 F137S

| | |
|---|---|
| Nucleotide | ATGAGCGCCCTGAGAAGAAAGTTCGGCGACGACTACCAGGTGGTCACC |
| sequence | ACAAGCAGCTCTGGCTCTGGACTGCAGCCTCAAGGACCTGGACAGGAT |
| | CCTCAGCAGCAGCTGGTGCCCAAGAAGAAACGGCAGAGATTCGTGGAC |
| | AAGAACGGCCGGTGCAATGTGCAGCACGGCAATCTGGGCAGCGAGACA |
| | AGCAGATACCTGAGCGACCTGTTCACCACACTGGTGGACCTGAAGTGG |
| | CGGTGGAACCTGTTCATCTTCATCCTGACCTACACCGTGGCCTGGCTGT |
| | TCATGGCCAGCATGTGGTGGGTTATCGCCTACACCAGAGGCGACCTGA |
| | ACAAGGCCCATGTGGGCAACTACACCCCTTGCGTGGCCAACGTGTACA |
| | ACTTCCCTAGCGCCTTCCTGTTCAGCATCGAGACAGAGGCCACAATCGG |
| | CTACGGCTACCGGTACATCACCGACAAGTGCCCCGAGGGAATTATCCTG |
| | TTCCTGTTCCAGTCCATCCTGGGCTCTATCGTGGATGCCTTCCTGATCG |
| | GCTGCATGTTCATCAAGATGAGCCAGCCTAAGAAGCGGGCCGAGACAC |
| | TGATGTTTAGCGAGCACGCCGTGATCAGCATGCGCGACGGAAAACTGA |
| | CCCTGATGTTCAGAGTGGGCAACCTGCGGAACAGCCACATGGTGTCAG |
| | CCCAGATCAGATGCAAGCTGCTGAAGTCCAGACAGACCCCTGAGGGCG |
| | AGTTCCTGCCTCTGGATCAGCTGGAACTGGACGTGGGCTTTAGCACAG |
| | GCGCCGATCAGCTGTTTCTGGTGTCCCCTCTGACCATCTGCCACGTGAT |
| | CGACGCTAAGAGCCCCTTCTACGATCTGAGCCAGCGGAGCATGCAGAC |
| | CGAGCAGTTTGAGATCGTGGTCATCCTGGAAGGCATTGTGGAAACCACC |
| | GGCATGACCTGCCAGGCCAGAACCAGCTACACAGAGGATGAGGTGCTG |
| | TGGGGCCACAGATTCTTCCCCGTGATCTCTCTGGAAGAGGGCTTCTTCA |
| | AGGTGGACTACAGCCAGTTCCACGCCACCTTTGAGGTGCCCACACCTC |
| | CTTACAGCGTGAAAGAACAAGAGGAAATGCTGCTGATGAGCAGCCCTCT |
| | GATCGCCCCTGCCATCACCAACAGCAAAGAACGGCACAACAGCGTGGA |
| | ATGCCTGGACGGCCTGGATGACATCACCACCAAGCTGCCTTCCAAGCT |
| | GCAGAAGATCACCGGCAGAGAGGACTTCCCCAAGAAACTGCTGAGAAT |
| | GAGCAGCACCACCAGCGAGAAGGCCTACTCTCTGGGCGACCTGCCTAT |
| | GAAGCTGCAGCGGATCTCTAGCGTGCCCGGCAACTCTGAGGAAAAGCT |
| | GGTTTCCAAGACCACCAAGATGCTGAGCGATCCCATGAGCCAGTCCGT |
| | GGCTGATCTGCCACCTAAACTGCAGAAAATGGCTGGCGGAGCCGCCAG |
| | AATGGAAGGCAATCTGCCTGCCAAACTGCGCAAGATGAACTCCGACCG |
| | GTTCACC (SEQ ID NO: 3) |
| Amino | MSALRRKFGDDYQVVTTSSSGSGLQPQGPGQDPQQQLVPKKKRQRFVDK |
| acid | NGRCNVQHGNLGSETSRYLSDLFTTLVDLKWRWNLFIFILTYTVAWLFMAS |
| sequence | MWWVIAYTRGDLNKAHVGNYTPCVANVYNFPSAFLFS₁₃₇IETEATIGYGYRY |
| (501 Aa) | ITDKCPEGIILFLFQSILGSIVDAFLIGCMFIKMSQPKKRAETLMFSEHAVI |
| | SMRDGKLTLMFRVGNLRNSHMVSAQIRCKLLKSRQTPEGEFLPLDQLELDVG |
| | FSTGADQLFLVSPLTICHVIDAKSPFYDLSQRSMQTEQFEIVVILEGIVETT |
| | GMTCQARTSYTEDEVLWGHRFFPVISLEEGFFKVDYSQFHATFEVPTPPYSV |
| | KEQEEMLLMSSPLIAPAITNSKERHNSVECLDGLDDITTKLPSKLQKITGRE |
| | DFPKKLLRMSSTTSEKAYSLGDLPMKLQRISSVPGNSEEKLVSKTTKMLSDP |
| | MSQSVADLPPKLQKMAGGAARMEGNLPAKLRKMNSDRFT (SEQ ID NO: 2) |

Mouse wild-type GIRK1

| | |
|---|---|
| Amino | MSALRRKFGDDYQVVTTSSSGSGLQPQGPGQGPQQQLVPKKKRQRFVDK |
| acid | NGRCNVQHGNLGSETSRYLSDLFTTLVDLKWRWNLFIFILTYTVAWLFMAS |
| sequence | MWWVIAYTRGDLNKAHVGNYTPCVANVYNFPSAFLFF₁₃₇IETEATIGYGYRY |
| (501 Aa) | ITDKCPEGIILFLFQSILGSIVDAFLIGCMFIKMSQPKKRAETLMFSEHAVI |
| | SMRDGKLTLMFRVGNLRNSHMVSAQIRCKLLKSRQTPEGEFLPLDQLELDVG |
| | FSTGADQLFLVSPLTICHVIDAKSPFYDLSQRSMQTEQFEVVVILEGIVETT |
| | GMTCQARTSYTEDEVLWGHRFFPVISLEEGFFKVDYSQFHATFEVPTPPYSV |
| | KEQEEMLLMSSPLIAPAITNSKERHNSVECLDGLDDISTKLPSKLQKITGRE |
| | DFPKKLLRMSSTTSEKAYSLGDLPMKLQRISSVPGNSEEKLVSKTTKMLSDP |
| | MSQSVADLPPKLQKMAGGPTRMEGNLPAKLRKMNSDRFT (SEQ ID NO: 4) |

Mouse GIRK1 F137S

| | |
|---|---|
| Amino | MSALRRKFGDDYQVVTTSSSGSGLQPQGPGQGPQQQLVPKKKRQRFVDKN |
| acid | GRCNVQHGNLGSETSRYLSDLFTTLVDLKWRWNLFIFILTYTVAWLFMASM |
| sequence | WWVIAYTRGDLNKAHVGNYTPCVANVYNFPSAFLFS₁₃₇IETEATIGYGYRY |
| (501 Aa) | ITDKCPEGIILFLFQSILGSIVDAFLIGCMFIKMSQPKKRAETLMFSEHAV |
| | ISMRDGKLTLMFRVGNLRNSHMVSAQIRCKLLKSRQTPEGEFLPLDQLELD |
| | VGFSTGADQLFLVSPLTICHVIDAKSPFYDLSQRSMQTEQFEVVVILEGIV |
| | ETTGMTCQARTSYTEDEVLWGHRFFPVISLEEGFFKVDYSQFHATFEVPTP |
| | PYSVKEQEEMLLMSSPLIAPAITNSKERHNSVECLDGLDDISTKLPSKLQK |
| | ITGREDFPKKLLRMSSTTSEKAYSLGDLPMKLQRISSVPGNSEEKLVSKTT |
| | KMLSDPMSQSVADLPPKLQKMAGGPTRMEGNLPAKLRKMNSDRFT |
| | (SEQ ID NO: 5) |

Another object of the present invention is a pharmaceutically acceptable carrier including a vector of the present invention.

According to a particular embodiment of the present invention, the pharmaceutically acceptable carrier can include a vector comprising a nucleotide sequence encoding GIRK1 F137S as described above and a vector comprising a nucleotide sequence encoding a mammalian cone opsin. For example, the mammalian cone opsin is a short wavelength cone opsin (SWO), e.g. from Mus musculus or human long wavelength-sensitive (OPN1LW), medium wavelength-sensitive (OPN1MW), short wavelength-sensitive (OPN1SW). According to an embodiment, the mammalian cone opsin is human Long-wave-sensitive opsin 1 (SEQ ID NO: 6).

Long-wave-sensitive opsin 1
(OPN1LW) homo sapiens

MVLKAEHTRSPSATLPSNVPSCRSLSSSEDGPSGPSSLADGGLAH
NLQDSVRHRILYLSEQLRVEKASRDGNTVSYLKLVSKADRHQVPH
IQQAFEKVNQRASATIAQIEHRLHQCHQQLQELEEGCRPEGLLLM
AESDPANCEPPSEKALLSEPPEPGGEDGPVNLPHASRPFILESRF
QSLQQGTCLETEDVAQQQNLLLQKVKAELEEAKRFHISLQESYHS
LKERSLTDLQLLLESLQEEKCRQALMEEQVNGRLQGQLNEIYNLK
HNLACSEERMAYLSYERAKEIWEITETFKSRISKLEMLQQVTQLE
AAEHLQSRPPQMLFKFLSPRLSLATVLLVFVSTLCACPSSLISSR
LCTCTMLMLIGLGVLAWQRWRAIPATDWQEWVPSRCRLYSKDSGP
PADGP
(SEQ ID NO: 6)

According to a particular embodiment of the present invention, the pharmaceutically acceptable carrier is for example chosen from solid-lipid nanoparticles, chitosan nanoparticles, liposome, lipoplex or cationic polymer.

According to a particular embodiment of the present invention, the vector of the present invention is a virus, chosen from an adeno-associated virus (AAV), an adenovirus, a lentivirus, an SV40 viral vector. According to a particular embodiment of the present invention, the present invention is equal to or less than 30 nm in size. For example it is an adeno-associated virus (AAV), preferably an AAV8, or an AAV2-7m8 or AAV9-7m8 capsid variant as described in the international patent application WO 2012/145601.

An AAV2-7m8 or AAV9-7m8 capsid variant is an AAV2 or AAV9 virus comprising a 7 to 11 amino acid long insertion peptide in the GH loop of the VP1 capsid protein, wherein the insertion peptide comprises amino acid sequence LGETTRP (SEQ ID NO: 7).

The genomic and polypeptide sequences of various serotypes of AAV, as well as the sequences of the native inverted terminal repeats (ITRs), Rep proteins, and capsid subunits including VP1 protein are known in the art. Such sequences may be found in the literature or in public databases such as GenBank or Protein Data Bank (PDB). See, e.g., GenBank and PDB AF043303 and 1LP3 (AAV2), AY530579 and 3UX1 (AAV9 (isolate hu.14)), the disclosures of which are incorporated by reference herein for teaching AAV nucleic acid and amino acid sequences. Exemplary amino acid sequence of wild-type VP1 for AAV9 and AAV2 are shown in SEQ ID NO: 8 and SEQ ID NO:9, respectively.

wild-type AAV9 VP1 capsid protein
MAADGYLPDWLEDNLSEGIREWWALKPGAPQPKANQQHQDNARGL
VLPGYKYLGPGNGLDKGEPVNAADAAALEHDKAYDQQLKAGDNPY
LKYNHADAEFQERLKEDTSFGGNLGRAVFQAKKRLLEPLGLVEEA
AKTAPGKKRPVEQSPQEPDSSAGIGKSGAQPAKKRLNFGQTGDTE
SVPDPQPIGEPPAAPSGVGSLTMASGGGAPVADNNEGADGVGSSS
GNWHCDSQWLGDRVITTSTRTWALPTYNNHLYKQISNSTSGGSSN
DNAYGYSTPWGYFDFNRFHCHFSPRDWQRLINNNWGFRPKRLNFK
LFNIQVKEVTDNNGVKTIANNLTSTVQVFTDSDYQLPYVLGSAHE
GCLPPFPADVFMIPQYGYLTLNDGSQAVGRSSFYCLEYFPSQMLR
TGNNFQFSYEFENVPFHSSYAHSQSLDRLMNPLIDQYLYYLSKTI
NGSGQNQQTLKFSVAGPSNMAVQGRNYIPGPSYRQQRVSTTVTQN
NNSEFAWPGASSWALNGRNSLMNPGPAMASHKEGEDRFFPLSGSL
IFGKQGTGRDNVDADKVMITNEEEIKTTNPVATESYGQVATNHQS
AQ588Q589AQTGWVQNQGILPGMVWQDRDVYLQGPIWAKIPHTD
GNFHPSPLMGGFGMKHPPPQILIKNTPVPADPPTAFNKDKLNSFI
TQYSTGQVSVEIEWELQKENSKRWNPEIQYTSNYYKSNNVEFAVN
TEGVYSEPRPIGTRYLTR
(SEQ ID NO: 8)
wild-type AAV2 VP1 capsid protein
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYYLSRTNT
PSGTTTQSRLQFSQAGASDIRDQSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GN587R588QAATADVNTQGVLPGMVWQDRDVYLQGPIWAKIPHT
DGHFHPSPLMGGFGLKHPPPQILIKNTPVPANPSTTFSAAKFASF
ITQYSTGQVSVEIEWELQKENSKRWNPEIQYTSNYNKSVNVDFTV
DTNGVYSEPRPIGTRYLTRNL
(SEQ ID NO: 9)

Preferably, the insertion site of the insertion peptide in the GH loop of the VP1 capsid protein is between amino acids 587 and 588 of AAV2 wild-type VP1 capsid protein, between amino acids 588 and 589 of AAV9 wild-type VP1 capsid protein.

According to some embodiments, the insertion peptide has a length of 7 amino acids, 8 amino acids, 9 amino acids, 10 amino acids, or 11 amino acids.

The insertion peptide may comprise one or more spacer amino acids at the N- and/or C-terminus of amino acid sequence LGETTRP (SEQ ID NO: 7). Preferably, the spacer amino acids are selected from the group consisting of Ala, Leu, Gly, Ser, and Thr, more preferably from the group consisting of Ala, Leu, and Gly.

According to an embodiment, the insertion peptide comprises or consists of sequence AALGETTRPA (SEQ ID NO: 10), LALGETTRPA (SEQ ID NO: 11), or GLGETTRPA (SEQ ID NO: 12), preferably comprises or consists of sequence AALGETTRPA (SEQ ID NO: 10) or LALGETTRPA (SEQ ID NO: 11).

According to a particular embodiment, the viral vector, in particular AAV, AAV8, AAV2-7m8 or AAV9-7m8, comprises the polynucleotide of interest (nucleotide sequence encoding GIRK1 F137S or a functional derivative thereof, and/or nucleotide sequence encoding mammalian cone opsin) under the control of a cone-specific promoter, preferably a pR1.7 or a functional variant thereof, or a minimal M-opsin promoter, in particular in a pMNTC expression cassette. In said AAV, the polynucleotide of interest which is operatively linked to the cone-specific promoter, e.g. promoter pR1.7, minimal M-opsin promoter or pMNTC, is preferably flanked by two adeno-associated virus inverted terminal repeats (AAV ITRs), preferably AAV2 ITRs.

pR1.7 is a 1.7 kilobases synthetic promoter based on the human red opsin promoter sequence described in Hum Gene Ther. 2016 January; 27 (1): 72-82. As used herein, “pR1.7” denotes the promoter of sequence SEQ ID NO:13 and functional variants thereof. “Functional variants” of the pR7.1 promoter typically have one or more nucleotide mutations (such as a nucleotide deletion, addition, and/or substitution) relative to the native pR7.1 promoter (SEQ ID NO: 13), which do not significantly alter the transcription of the polynucleotide of interest. In the context of the present invention, said functional variants retain the capacity to drive a strong expression, in cone photoreceptors, of the polynucleotide of interest. Such capacity can be tested as described by Ye et al. (Hum. Gene Ther. 2016; 27 (1): 72-82) and Khabou et al. (JCI Insight. 2018, 3 (2): e96029).

Another example of cone-specific promoter which may be used is a minimal M-opsin promoter region such as disclosed in WO2015142941, in particular in SEQ ID NO:55 or SEQ ID NO: 93 as disclosed in WO2015142941. Instant sequence SEQ ID NO:14 is identical to SEQ ID NO: 93 of WO2015142941.

In an embodiment, the polynucleotide of interest which is placed under the control the minimal M-opsin promoter region, is inserted in a pMNTC expression cassette comprising an optimized enhancer, optimized promoter, optimized 5′UTR, optimized intron, optimized kozak and optimized polyA region (SEQ ID NO:95 of WO2015142941).

pR1.7 promoter
ggaggctgaggggggggaaagggcatgggtgtttcatgaggacag
agcttccgtttcatgcaatgaaaagagtttggagacggatggtgg
tgactggactatacacttacacacggtagcgatggtacactttgt
attatgtatattttaccacgatctttttaaagtgtcaaaggcaaa
tggccaaatggttccttgtcctatagctgtagcagccatcggctg
ttagtgacaaagcccctgagtcaagatgacagcagcccccataac
tcctaatcggctctcccgcgtggagtcatttaggagtagtcgcat
tagagacaagtccaacatctaatcttccaccctggccagggcccc
agctggcagcgaggggggagactccgggcagagcagagggcgctg
acattggggcccggcctggcttgggtccctctggcctttccccag
gggccctctttccttggggctttcttgggccgccactgctcccgc
tcctctccccccatcccaccccctcaccccctcgttcttcatatc
cttctctagtgctccctccactttcatccacccttctgcaagagt
gtgggaccacaaatgagttttcacctggcctggggacacacgtgc
ccccacaggtgctgagtgactttctaggacagtaatctgctttag
gctaaaatgggacttgatcttctgttagccctaatcatcaattag
cagagccggtgaaggtgcagaacctaccgcctttccaggcctcct
cccacctctgccacctccactctccttcctgggatgtgggggctg
gcacacgtgtggcccagggcattggtgggattgcactgagctggg
tcattagcgtaatcctggacaagggcagacagggcgagcggaggg
ccagctccggggctcaggcaaggctgggggcttcccccagacacc
ccactcctcctctgctggacccccacttcatagggcacttcgtgt
tctcaaagggcttccaaatagcatggtggccttggatgcccaggg
aagcctcagagttgcttatctccctctagacagaaggggaatctc
ggtcaagagggagaggtcgccctgttcaaggccacccagccagct
catggcggtaatgggacaaggctggccagccatcccaccctcaga
agggacccggtggggcaggtgatctcagaggaggctcacttctgg
gtctcacattcttggatccggttccaggcctcggccctaaatagt
ctccctgggctttcaagagaaccacatgagaaaggaggattcggg
ctctgagcagtttcaccacccaccccccagtctgcaaatcctgac
ccgtgggtccacctgccccaaaggcggacgcaggacagtagaagg
gaacagagaacacataaacacagagagggccacagcggctcccac
agtcaccgccaccttcctggcggggatgggtggggcgtctgagtt
tggttcccagcaaatccctctgagccgcccttgcgggctcgcctc
aggagcaggggagcaagaggtgggaggaggaggtctaagtcccag
gcccaattaagagatcaggtagtgtagggtttgggagcttttaag
gtgaagaggcccgggctgatcccacaggccagtataaagcgccgt
gaccctcaggtgatgcgccagggccggctgccgtcggggacaggg
ctttccatagcc
(SEQ ID NO: 13)
minimal M-opsin promoter region
Ccagcaaatccctctgagccgcccccgggggctcgcctcaggagc
aaggaagcaaggggtgggaggaggaggtctaagtcccaggcccaa
ttaagagatcagatggtgtaggatttgggagcttttaaggtgaag
aggcccgggctgatcccactggccggtataaagcaccgtgaccct
caggtgacgcaccagggccggctgccgtcggggacagggctttcc
atagcccag
(SEQ ID NO: 14)
pMNTC
cctacagcagccagggtgagattatgaggctgagctgagaatatc
aagactgtaccgagtagggggccttggcaagtgtggagagcccgg
cagctggggcagagggcggagtacggtgtgcgtttacggacctct
tcaaacgaggtaggaaggtcagaagtcaaaaagggaacaaatgat
gtttaaccacacaaaaatgaaaatccaatggttggatatccattc
caaatacacaaaggcaacggataagtgatccgggccaggcacaga
aggccatgcacccgtaggattgcactcagagctcccaaatgcata
ggaatagaagggtgggtgcaggaggctgaggggggggaaagggca
tgggtgtttcatgaggacagagcttccgtttcatgcaatgaaaag
agtttggagacggatggtggtgactggactatacacttacacacg
gtagcgatggtacactttgtattatgtatattttaccacgatctt
tttaaagtgtcaaaggcaaatggccaaatggttccttgtcctata
gctgtagcagccatcggctgttagtgacaaagcccctgagtcaag
atgacagcagcccccataactcctaatcggctctcccgcgtggag
tcatttaggagtagtcgcattagagacaagtccaacatctaatct
tccaccctggccagggccccagctggcagcgaggggggagactcc
gggcagagcagagggcgctgacattggggcccggcctggcttggg
tccctctggcctttccccaggggccctctttccttggggctttct
tgggccgccactgctcccgctcctctccccccatcccaccccctc
accccctcgttcttcatatccttctctagtgctccctccactttc
atccacccttctgcaagagtgtgggaccacaaatgagttttcacc
tggcctggggacacacgtgcccccacaggtgctgagtgactttct
aggacagtaatctgctttaggctaaaatgggacttgatcttctgt
tagccctaatcatcaattagcagagccggtgaaggtgcagaacct
accgcctttccaggcctcctcccacctctgccacctccactctcc
ttcctgggatgtgggggctggcacacgtgtggcccagggcattgg
tgggattgcactgagctgggtcattagcgtaatcctggacaaggg
cagacagggcgagcggagggccagctccggggctcaggcaaggct
gggggcttcccccagacaccccactcctcctctgctggaccccca
cttcatagggcacttcgtgttctcaaagggcttccaaatagcatg
gtggccttggatgcccagggaagcctcagagttgcttatctccct
ctagacagaaggggaatctcggtcaagagggagaggtcgccctgt
tcaaggccacccagccagctcatggcggtaatgggacaaggctgg
ccagccatcccaccctcagaagggacccggtggggcaggtgatct
cagaggaggctcacttctgggtctcacattcttccagcaaatccc
tctgagccgcccccgggggctcgcctcaggagcaaggaagcaagg
ggtgggaggaggaggtctaagtcccaggcccaattaagagatcag
atggtgtaggatttgggagcttttaaggtgaagaggcccgggctg
atcccactggccggtataaagcaccgtgaccctcaggtgacgcac
cagggccggctgccgtcggggacagggctttccatagcccaggta
agtatcaaggttacaagacaggtttaaggagaccaatagaaactg
ggcttgtcgagacagagaagactcttgcgtttctgataggcacct
attggtcttactgacatccactttgcctttctctccacaggccca
gagaggagacaggccgccacc
(SEQ ID NO: 15)

The promoter and the polynucleotide of interest are operatively linked. As used herein, the term “operably linked” refers to two or more nucleic acid or amino acid sequence elements that are physically linked in such a way that they are in a functional relationship with each other. For instance, a promoter is operably linked to a coding sequence if the promoter is able to initiate or otherwise control/regulate the transcription and/or expression of a coding sequence, in which case the coding sequence should be understood as being “under the control of” the promoter. Generally, when two nucleic acid sequences are operably linked, they will be in the same orientation and usually also in the same reading frame. They will usually also be essentially contiguous, although this may not be required.

According to an embodiment, the vector is an AAV9 (AAV9-7m8-pR1.7) or an AAV2 (AAV2-7m8-pR1.7) comprising:
a VP1 capsid protein in which a 7 to 11 amino acid long insertion peptide is inserted in the GH loop of said VP1 capsid protein relative to wild-type AAV9 VP1 capsid protein, at a position localized between amino acids 588 and 589 of wild-type AAV9 VP1 capsid protein, wherein said peptide comprises amino acid sequence LGETTRP (SEQ ID NO: 7); and
the polynucleotide of interest (nucleotide sequence encoding GIRK1 F137S and/or nucleotide sequence encoding mammalian cone opsin) under the control of a pR1.7 promoter.

In said AAV9-7m8 or AAV2-7m8, the insertion peptide has a length of 7 amino acids, 8 amino acids, 9 amino acids, 10 amino acids, or 11 amino acids. Preferably, the insertion peptide comprises one or more spacer amino acids at the N- and/or C-terminus of amino acid sequence LGETTRP (SEQ ID NO: 7). Preferably, the spacer amino acids are selected from the group consisting of Ala, Leu, Gly, Ser, and Thr, more preferably from the group consisting of Ala, Leu, and Gly. According to an embodiment, the insertion peptide comprises or consists of sequence AALGETTRPA (SEQ ID NO: 10), LALGETTRPA (SEQ ID NO: 11), or GLGETTRPA (SEQ ID NO: 12); preferably comprises or consists of sequence AALGETTRPA (SEQ ID NO: 10) or LALGETTRPA (SEQ ID NO: 11).

The vectors of the invention are produced using methods known in the art. In short, the methods generally involve (a) the introduction of the AAV vector into a host cell, (b) the introduction of an AAV helper construct into the host cell, wherein the helper construct comprises the viral functions missing from the AAV vector and (c) introducing a helper virus into the host cell. All functions for AAV virion replication and packaging need to be present, to achieve replication and packaging of the AAV vector into AAV virions. The introduction into the host cell can be carried out using standard virology techniques simultaneously or sequentially. Finally, the host cells are cultured to produce AAV virions and are purified using standard techniques such as iodixanol or CsCl gradients or other purification methods. The purified AAV virion is then ready for use.

Another object of the present invention is a nucleic acid comprising a nucleotide sequence encoding GIRK1 F137S as described above, for use as a medicament. In particular, said nucleic acid is for use in treating rod-cone dystrophy (RCD). In other words, another object of the present invention is a method of treating a RCD in a mammal in need thereof, the method comprising administering to the mammal a nucleic acid comprising a nucleotide sequence encoding GIRK1 F137S as described above.

In an embodiment, the nucleic acid comprising a nucleotide sequence encoding GIRK1 F137S comprises or consists of a nucleotide sequence encoding the polypeptide of sequence SEQ ID NO: 2, or comprises or consists of the nucleotide sequence SEQ ID NO: 3.

The nucleic acid comprising a nucleotide sequence encoding GIRK1 F137S may be in a vector selected from the group consisting of an adeno-associated virus (AAV), an adenovirus, a lentivirus, and SV40 viral vector.

According to an embodiment, the nucleic acid comprising a nucleotide sequence encoding GIRK1 F137S is under the control of the pR1.7 promoter or of a functional variant of said promoter.

According to an embodiment, the nucleic acid comprising a nucleotide sequence encoding GIRK1 F137S is in a vector which is an AAV2 or AVV9 virus comprising a 7 to 11 amino acid long insertion peptide in the GH loop of the VP1 capsid protein, wherein the insertion peptide comprises amino acid sequence LGETTRP (SEQ ID NO: 7).

Preferably, the vector is a recombinant AAV2 or AAV9 vector comprising:
a VP1 capsid protein in a 7 to 11 amino acid long insertion peptide is inserted in the GH loop of said VP1 capsid protein relative to wild-type AAV9 VP1 capsid protein, at a position localized between amino acids 588 and 589 of wild-type AAV9 VP1 capsid protein, wherein said peptide comprises amino acid sequence LGETTRP (SEQ ID NO: 7); and
the nucleotide sequence encoding the mammalian cone opsin under the control of a pR1.7 promoter.

According to an embodiment, said insertion peptide comprises or consists of amino acid sequence AALGETTRPA (SEQ ID NO: 8), LALGETTRPA (SEQ ID NO: 9), or GLGETTRPA (SEQ ID NO: 10).

According to an embodiment, the nucleic acid comprising a nucleotide sequence encoding GIRK1 F137S further comprises a sequence encoding a mammalian cone opsin, as described above.

According to another embodiment, the nucleic acid comprising a nucleotide sequence encoding GIRK1 F137S is for use in treating RCD in combination with another nucleic acid encoding a mammalian cone opsin, as described above. In other words, another object of the present invention is a method of treating a RCD in a mammal in need thereof, the method comprising administering to the mammal a nucleic acid comprising a nucleotide sequence encoding GIRK1 F137S and another nucleic acid encoding a mammalian cone opsin. In said method, the nucleic acids are therefore in separate forms and may be formulated in a single pharmaceutical composition or in separate pharmaceutical compositions.

In one embodiment, the nucleic acid comprising a nucleotide sequence encoding GIRK1 F137S comprises the expression cassette from plasmid pAAV2-5′ITR-pR1.7-hGIRK-F137S-BGHpA-AAV2-3′ITR (having the sequence as set forth in SEQ ID NO:16), pAAV2-5′ITR-pR1.7-hGIRK-F137S-WPREmut6-BGHpA-AAV2-3′ITR (having the sequence as set forth in SEQ ID NO:17) or pAAV2-5′ITR-pR1.7-hGIRK-F137S-WPREmut6deltaATG-BGHpA-AAV2-3′ITR (having the sequence as set forth in SEQ ID NO:18).

pAAV2-5′ITR-pR1.7-hGIRK-F137S-BGHpA-AAV2-3′ITR
cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttccttctagacaa
ctttgtatagaaaagttgggaggctgaggggggggaaagggcatgggtgt
ttcatgaggacagagcttccgtttcatgcaatgaaaagagtttggagacg
gatggtggtgactggactatacacttacacacggtagcgatggtacactt
tgtattatgtatattttaccacgatctttttaaagtgtcaaaggcaaatg
gccaaatggttccttgtcctatagctgtagcagccatcggctgttagtga
caaagcccctgagtcaagatgacagcagcccccataactcctaatcggct
ctcccgcgtggagtcatttaggagtagtcgcattagagacaagtccaaca
tctaatcttccaccctggccagggccccagctggcagcgaggggggagac
tccgggcagagcagagggcgctgacattggggcccggcctggcttgggtc
cctctggcctttccccaggggccctctttccttggggctttcttgggccg
ccactgctcccgctcctctccccccatcccaccccctcaccccctcgttc
ttcatatccttctctagtgctccctccactttcatccacccttctgcaag
agtgtgggaccacaaatgagttttcacctggcctggggacacacgtgccc
ccacaggtgctgagtgactttctaggacagtaatctgctttaggctaaaa
tgggacttgatcttctgttagccctaatcatcaattagcagagccggtga
aggtgcagaacctaccgcctttccaggcctcctcccacctctgccacctc
cactctccttcctgggatgtgggggctggcacacgtgtggcccagggcat
tggtgggattgcactgagctgggtcattagcgtaatcctggacaagggca
gacagggcgagcggagggccagctccggggctcaggcaaggctgggggct
tcccccagacaccccactcctcctctgctggacccccacttcatagggca
cttcgtgttctcaaagggcttccaaatagcatggtggccttggatgccca
gggaagcctcagagttgcttatctccctctagacagaaggggaatctcgg
tcaagagggagaggtcgccctgttcaaggccacccagccagctcatggcg
gtaatgggacaaggctggccagccatcccaccctcagaagggacccggtg
gggcaggtgatctcagaggaggctcacttctgggtctcacattcttggat
ccggttccaggcctcggccctaaatagtctccctgggctttcaagagaac
cacatgagaaaggaggattcgggctctgagcagtttcaccacccaccccc
cagtctgcaaatcctgacccgtgggtccacctgccccaaaggcggacgca
ggacagtagaagggaacagagaacacataaacacagagagggccacagcg
gctcccacagtcaccgccaccttcctggcggggatgggtggggcgtctga
gtttggttcccagcaaatccctctgagccgcccttgcgggctcgcctcag
gagcaggggagcaagaggtgggaggaggaggtctaagtcccaggcccaat
taagagatcaggtagtgtagggtttgggagcttttaaggtgaagaggccc
gggctgatcccacaggccagtataaagcgccgtgaccctcaggtgatgcg
ccagggccggctgccgtcggggacagggctttccatagccactagtgtaa
gtttagtctttttgtcttttatttcaggtcccggatccggtggtggtgca
aatcaaagaactgctcctcagtggatgttgcctttacttctagcaagttt
gtacaaaaaagcaggctgccaccatgagcgccctgagaagaaagttcggc
gacgactaccaggtggtcaccacaagcagctctggctctggactgcagcc
tcaaggacctggacaggatcctcagcagcagctggtgcccaagaagaaac
ggcagagattcgtggacaagaacggccggtgcaatgtgcagcacggcaat
ctgggcagcgagacaagcagatacctgagcgacctgttcaccacactggt
ggacctgaagtggcggtggaacctgttcatcttcatcctgacctacaccg
tggcctggctgttcatggccagcatgtggtgggttatcgcctacaccaga
ggcgacctgaacaaggcccatgtgggcaactacaccccttgcgtggccaa
cgtgtacaacttccctagcgccttcctgttcagcatcgagacagaggcca
caatcggctacggctaccggtacatcaccgacaagtgccccgagggaatt
atcctgttcctgttccagtccatcctgggctctatcgtggatgccttcct
gatcggctgcatgttcatcaagatgagccagcctaagaagcgggccgaga
cactgatgtttagcgagcacgccgtgatcagcatgcgcgacggaaaactg
accctgatgttcagagtgggcaacctgcggaacagccacatggtgtcagc
ccagatcagatgcaagctgctgaagtccagacagacccctgagggcgagt
tcctgcctctggatcagctggaactggacgtgggctttagcacaggcgcc
gatcagctgtttctggtgtcccctctgaccatctgccacgtgatcgacgc
taagagccccttctacgatctgagccagcggagcatgcagaccgagcagt
ttgagatcgtggtcatcctggaaggcattgtggaaaccaccggcatgacc
tgccaggccagaaccagctacacagaggatgaggtgctgtggggccacag
attcttccccgtgatctctctggaagagggcttcttcaaggtggactaca
gccagttccacgccacctttgaggtgcccacacctccttacagcgtgaaa
gaacaagaggaaatgctgctgatgagcagccctctgatcgcccctgccat
caccaacagcaaagaacggcacaacagcgtggaatgcctggacggcctgg
atgacatcaccaccaagctgccttccaagctgcagaagatcaccggcaga
gaggacttccccaagaaactgctgagaatgagcagcaccaccagcgagaa
ggcctactctctgggcgacctgcctatgaagctgcagcggatctctagcg
tgcccggcaactctgaggaaaagctggtttccaagaccaccaagatgctg
agcgatcccatgagccagtccgtggctgatctgccacctaaactgcagaa
aatggctggcggagccgccagaatggaaggcaatctgcctgccaaactgc
gcaagatgaactccgaccggttcacctagacccagctttcttgtacaaag
tgggaattcctagagctcgctgatcagcctcgactgtgccttctagttgc
cagccatctgttgtttgcccctcccccgtgccttccttgaccctggaagg
tgccactcccactgtcctttcctaataaaatgaggaaattgcatcgcatt
gtctgagtaggtgtcattctattctggggggtggggtggggcaggacagc
aagggggaggattgggaagagaatagcaggcatgctggggagggccgcag
gaacccctagtgatggagttggccactccctctctgcgcgctcgctcgct
cactgaggccgggcgaccaaaggtcgcccgacgcccgggctttgcccggg
cggcctcagtgagcgagcgagcgcgcagctgcctgcaggggcgcctgatg
cggtattttctccttacgcatctgtgcggtatttcacaccgcatacgtca
aagcaaccatagtacgcgccctgtagcggcgcattaagcgcggcgggtgt
ggtggttacgcgcagcgtgaccgctacacttgccagcgccttagcgcccg
ctcctttcgctttcttcccttcctttctcgccacgttcgccggctttccc
cgtcaagctctaaatcgggggctccctttagggttccgatttagtgcttt
acggcacctcgaccccaaaaaacttgatttgggtgatggttcacgtagtg
ggccatcgccctgatagacggtttttcgccctttgacgttggagtccacg
ttctttaatagtggactcttgttccaaactggaacaacactcaactctat
ctcgggctattttttgatttataagggattttgccgatttcggtctattg
gttaaaaaatgagctgatttaacaaaaatttaacgcgaattttaacaaaa
tattaacgtttacaattttatggtgcactctcagtacaatctgctctgat
gccgcatagttaagccagccccgacacccgccaacacccgctgacgcgcc
ctgacgggcttgtctgctcccggcatccgcttacagacaagctgtgaccg
tctccgggagctgcatgtgtcagaggttttcaccgtcatcaccgaaacgc
gcgagacgaaagggcctcgtgatacgcctatttttataggttaatgtcat
gataataatggtttcttagacgtcaggtggcacttttcggggaaatgtgc
gcggaacccctatttgtttatttttctaaatacattcaaatatgtatccg
ctcatgagacaataaccctgataaatgcttcaataatattgaaaaaggaa
gagtatgagccatattcaacgggaaacgtcttgctctaggccgcgattaa
attccaacatggatgctgatttatatgggtataaatgggctcgcgataat
gtcgggcaatcaggtgcgacaatctatcgattgtatgggaagcccgatgc
gccagagttgtttctgaaacatggcaaaggtagcgttgccaatgatgtta
cagatgagatggtcagactaaactggctgacggaatttatgcctcttccg
accatcaagcattttatccgtactcctgatgatgcatggttactcaccac
tgcgatccccgggaaaacagcattccaggtattagaagaatatcctgatt
caggtgaaaatattgttgatgcgctggcagtgttcctgcgccggttgcat
tcgattcctgtttgtaattgtccttttaacagcgatcgcgtatttcgtct
cgctcaggcgcaatcacgaatgaataacggtttggttgatgcgagtgatt
ttgatgacgagcgtaatggctggcctgttgaacaagtctggaaagaaatg
cataaacttttgccattctcaccggattcagtcgtcactcatggtgattt
ctcacttgataaccttatttttgacgaggggaaattaataggttgtattg
atgttggacgagtcggaatcgcagaccgataccaggatcttgccatccta
tggaactgcctcggtgagttttctccttcattacagaaacggctttttca
aaaatatggtattgataatcctgatatgaataaattgcagtttcatttga
tgctcgatgagtttttctaactgtcagaccaagtttactcatatatactt
tagattgatttaaaacttcatttttaatttaaaaggatctaggtgaagat
cctttttgataatctcatgaccaaaatcccttaacgtgagttttcgttcc
actgagcgtcagaccccgtagaaaagatcaaaggatcttcttgagatcct
ttttttctgcgcgtaatctgctgcttgcaaacaaaaaaaccaccgctacc
agcggtggtttgtttgccggatcaagagctaccaactctttttccgaagg
taactggcttcagcagagcgcagataccaaatactgttcttctagtgtag
ccgtagttaggccaccacttcaagaactctgtagcaccgcctacatacct
cgctctgctaatcctgttaccagtggctgctgccagtggcgataagtcgt
gtcttaccgggttggactcaagacgatagttaccggataaggcgcagcgg
tcgggctgaacggggggttcgtgcacacagcccagcttggagcgaacgac
ctacaccgaactgagatacctacagcgtgagctatgagaaagcgccacgc
ttcccgaagggagaaaggcggacaggtatccggtaagcggcagggtcgga
acaggagagcgcacgagggagcttccagggggaaacgcctggtatcttta
tagtcctgtcgggtttcgccacctctgacttgagcgtcgatttttgtgat
gctcgtcaggggggcggagcctatggaaaaacgccagcaacgcggccttt
ttacggttcctggccttttgctggccttttgctcacatgt
(SEQ ID NO: 16)
pAAV2-5′ITR-pR1.7-hGIRK-F137S-WPREmut6-BGHpA-
AAV2-3′ITR
cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttccttctagacaa
ctttgtatagaaaagttgggaggctgaggggggggaaagggcatgggtgt
ttcatgaggacagagcttccgtttcatgcaatgaaaagagtttggagacg
gatggtggtgactggactatacacttacacacggtagcgatggtacactt
tgtattatgtatattttaccacgatctttttaaagtgtcaaaggcaaatg
gccaaatggttccttgtcctatagctgtagcagccatcggctgttagtga
caaagcccctgagtcaagatgacagcagcccccataactcctaatcggct
ctcccgcgtggagtcatttaggagtagtcgcattagagacaagtccaaca
tctaatcttccaccctggccagggccccagctggcagcgaggggggagac
tccgggcagagcagagggcgctgacattggggcccggcctggcttgggtc
cctctggcctttccccaggggccctctttccttggggctttcttgggccg
ccactgctcccgctcctctccccccatcccaccccctcaccccctcgttc
ttcatatccttctctagtgctccctccactttcatccacccttctgcaag
agtgtgggaccacaaatgagttttcacctggcctggggacacacgtgccc
ccacaggtgctgagtgactttctaggacagtaatctgctttaggctaaaa
tgggacttgatcttctgttagccctaatcatcaattagcagagccggtga
aggtgcagaacctaccgcctttccaggcctcctcccacctctgccacctc
cactctccttcctgggatgtgggggctggcacacgtgtggcccagggcat
tggtgggattgcactgagctgggtcattagcgtaatcctggacaagggca
gacagggcgagcggagggccagctccggggctcaggcaaggctgggggct
tcccccagacaccccactcctcctctgctggacccccacttcatagggca
cttcgtgttctcaaagggcttccaaatagcatggtggccttggatgccca
gggaagcctcagagttgcttatctccctctagacagaaggggaatctcgg
tcaagagggagaggtcgccctgttcaaggccacccagccagctcatggcg
gtaatgggacaaggctggccagccatcccaccctcagaagggacccggtg
gggcaggtgatctcagaggaggctcacttctgggtctcacattcttggat
ccggttccaggcctcggccctaaatagtctccctgggctttcaagagaac
cacatgagaaaggaggattcgggctctgagcagtttcaccacccaccccc
cagtctgcaaatcctgacccgtgggtccacctgccccaaaggcggacgca
ggacagtagaagggaacagagaacacataaacacagagagggccacagcg
gctcccacagtcaccgccaccttcctggcggggatgggtggggcgtctga
gtttggttcccagcaaatccctctgagccgcccttgcgggctcgcctcag
gagcaggggagcaagaggtgggaggaggaggtctaagtcccaggcccaat
taagagatcaggtagtgtagggtttgggagcttttaaggtgaagaggccc
gggctgatcccacaggccagtataaagcgccgtgaccctcaggtgatgcg
ccagggccggctgccgtcggggacagggctttccatagccactagtgtaa
gtttagtctttttgtcttttatttcaggtcccggatccggtggtggtgca
aatcaaagaactgctcctcagtggatgttgcctttacttctagcaagttt
gtacaaaaaagcaggctgccaccatgagcgccctgagaagaaagttcggc
gacgactaccaggtggtcaccacaagcagctctggctctggactgcagcc
tcaaggacctggacaggatcctcagcagcagctggtgcccaagaagaaac
ggcagagattcgtggacaagaacggccggtgcaatgtgcagcacggcaat
ctgggcagcgagacaagcagatacctgagcgacctgttcaccacactggt
ggacctgaagtggcggtggaacctgttcatcttcatcctgacctacaccg
tggcctggctgttcatggccagcatgtggtgggttatcgcctacaccaga
ggcgacctgaacaaggcccatgtgggcaactacaccccttgcgtggccaa
cgtgtacaacttccctagcgccttcctgttcagcatcgagacagaggcca
caatcggctacggctaccggtacatcaccgacaagtgccccgagggaatt
atcctgttcctgttccagtccatcctgggctctatcgtggatgccttcct
gatcggctgcatgttcatcaagatgagccagcctaagaagcgggccgaga
cactgatgtttagcgagcacgccgtgatcagcatgcgcgacggaaaactg
accctgatgttcagagtgggcaacctgcggaacagccacatggtgtcagc
ccagatcagatgcaagctgctgaagtccagacagacccctgagggcgagt
tcctgcctctggatcagctggaactggacgtgggctttagcacaggcgcc
gatcagctgtttctggtgtcccctctgaccatctgccacgtgatcgacgc
taagagccccttctacgatctgagccagcggagcatgcagaccgagcagt
ttgagatcgtggtcatcctggaaggcattgtggaaaccaccggcatgacc
tgccaggccagaaccagctacacagaggatgaggtgctgtggggccacag
attcttccccgtgatctctctggaagagggcttcttcaaggggactacag
ccagttccacgccacctttgaggtgcccacacctccttacagcgtgaaag
aacaagaggaaatgctgctgatgagcagccctctgatcgcccctgccatc
accaacagcaaagaacggcacaacagcgtggaatgcctggacggcctgga
tgacatcaccaccaagctgccttccaagctgcagaagatcaccggcagag
aggacttccccaagaaactgctgagaatgagcagcaccaccagcgagaag
gcctactctctgggcgacctgcctatgaagctgcagcggatctctagcgt
gcccggcaactctgaggaaaagctggtttccaagaccaccaagatgctga
gcgatcccatgagccagtccgtggctgatctgccacctaaactgcagaaa
atggctggcggagccgccagaatggaaggcaatctgcctgccaaactgcg
caagatgaactccgaccggttcacctagacccagctttcttgtacaaagt
gggaattcATCAACCTCTGGATTACAAAATTTGTGAAAGATTGACTGATA
TTCTTAACTATGTTGCTCCTTTTACGCTGTGTGGATATGCTGCTTTAATG
CCTCTGTATCATGCTATTGCTTCCCGTACGGCTTTCGTTTTCTCCTCCTT
GTATAAATCCTGGTTGCTGTCTCTTTATGAGGAGTTGTGGCCCGTTGTCC
GTCAACGTGGCGTGGTGTGCTCTGTGTTTATCATCGCAACCCCCACTGGC
TGGGGCATTGCCACCACCTGTCAACTCCTTTCTGGGACTTTCGCTTTCCC
CCTCCCGATCGCCACGGCAGAACTCATCGCCGCCTGCCTTGCCCGCTGCT
GGACAGGGGCTAGGTTGCTGGGCACTGATAATTCCGTGGTGTTGTCGGGG
AAGCTGACGTCCTTTCCTTGGCTGCTCGCCTGTGTTGCCAACTGGATCCT
GCGCGGGACGTCCTTCTGCTACGTCCCTTCGGCTCTCAATCCAGCGGACC
TCCCTTCCCGAGGCCTTCTGCCGGTTCTGCGGCCTCTCCCGCGTCTTCGC
TTTCGGCCTCCGACGAGTCGGATCTCCCTTTGGGCCGCCTCCCCGCCTGc
tagagctcgctgatcagcctcgactgtgccttctagttgccagccatctg
ttgtttgcccctcccccgtgccttccttgaccctggaaggtgccactccc
actgtcctttcctaataaaatgaggaaattgcatcgcattgtctgagtag
gtgtcattctattctggggggggggtggggcaggacagcaagggggagga
ttgggaagagaatagcaggcatgctggggagggccgcaggaaCCCCtagt
gatggagttggccactccctctctgcgcgctcgctcgctcactgaggccg
ggcgaccaaaggtcgcccgacgcccgggctttgcccgggcggcctcagtg
agcgagcgagcgcgcagctgcctgcaggggcgcctgatgcggtattttct
ccttacgcatctgtgcggtatttcacaccgcatacgtcaaagcaaccata
gtacgcgccctgtagcggcgcattaagcgcggcgggtgtggtggttacgc
gcagcgtgaccgctacacttgccagcgccttagcgcccgctcctttcgct
ttcttcccttcctttctcgccacgttcgccggctttccccgtcaagctct
aaatcgggggctccctttagggttccgatttagtgctttacggcacctcg
accccaaaaaacttgatttgggtgatggttcacgtagtgggccatcgccc
tgatagacggtttttcgccctttgacgttggagtccacgttctttaatag
tggactcttgttccaaactggaacaacactcaactctatctcgggctatt
cttttgatttataagggattttgccgatttcggtctattggttaaaaaat
gagctgatttaacaaaaatttaacgcgaattttaacaaaatattaacgtt
tacaattttatggtgcactctcagtacaatctgctctgatgccgcatagt
taagccagccccgacacccgccaacacccgctgacgcgccctgacgggct
tgtctgctcccggcatccgcttacagacaagctgtgaccgtctccgggag
ctgcatgtgtcagaggttttcaccgtcatcaccgaaacgcgcgagacgaa
agggcctcgtgatacgcctatttttataggttaatgtcatgataataatg
gtttcttagacgtcaggtggcacttttcggggaaatgtgcgcggaacccc
tatttgtttatttttctaaatacattcaaatatgtatccgctcatgagac
aataaccctgataaatgcttcaataatattgaaaaaggaagagtatgagc
catattcaacgggaaacgtcttgctctaggccgcgattaaattccaacat
ggatgctgatttatatgggtataaatgggctcgcgataatgtcgggcaat
caggtgcgacaatctatcgattgtatgggaagcccgatgcgccagagttg
tttctgaaacatggcaaaggtagcgttgccaatgatgttacagatgagat
ggtcagactaaactggctgacggaatttatgcctcttccgaccatcaagc
attttatccgtactcctgatgatgcatggttactcaccactgcgatcccc
gggaaaacagcattccaggtattagaagaatatcctgattcaggtgaaaa
tattgttgatgcgctggcagtgttcctgcgccggttgcattcgattcctg
tttgtaattgtccttttaacagcgatcgcgtatttcgtctcgctcaggcg
caatcacgaatgaataacggtttggttgatgcgagtgattttgatgacga
gcgtaatggctggcctgttgaacaagtctggaaagaaatgcataaacttt
tgccattctcaccggattcagtcgtcactcatggtgatttctcacttgat
aaccttatttttgacgaggggaaattaataggttgtattgatgttggacg
agtcggaatcgcagaccgataccaggatcttgccatcctatggaactgcc
tcggtgagttttctccttcattacagaaacggctttttcaaaaatatggt
attgataatcctgatatgaataaattgcagtttcatttgatgctcgatga
gtttttctaactgtcagaccaagtttactcatatatactttagattgatt
taaaacttcatttttaatttaaaaggatctaggtgaagatcctttttgat
aatctcatgaccaaaatcccttaacgtgagttttcgttccactgagcgtc
agaccccgtagaaaagatcaaaggatcttcttgagatcctttttttctgc
gcgtaatctgctgcttgcaaacaaaaaaaccaccgctaccagcggtggtt
tgtttgccggatcaagagctaccaactctttttccgaaggtaactggctt
cagcagagcgcagataccaaatactgttcttctagtgtagccgtagttag
gccaccacttcaagaactctgtagcaccgcctacatacctcgctctgcta
atcctgttaccagtggctgctgccagtggcgataagtcgtgtcttaccgg
gttggactcaagacgatagttaccggataaggcgcagcggtcgggctgaa
cggggggttcgtgcacacagcccagcttggagcgaacgacctacaccgaa
ctgagatacctacagcgtgagctatgagaaagcgccacgcttcccgaagg
gagaaaggcggacaggtatccggtaagcggcagggtcggaacaggagagc
gcacgagggagcttccagggggaaacgcctggtatctttatagtcctgtc
gggtttcgccacctctgacttgagcgtcgatttttgtgatgctcgtcagg
ggggcggagcctatggaaaaacgccagcaacgcggcctttttacggttcc
tggccttttgctggccttttgctcacatgt
(SEQ ID NO: 17)
pAAV2-5′ITR-pR1.7-hGIRK-F137S-WPREmut6deltaATG-
BGHpA-AAV2-3′ITR
cctgcaggcagctgcgcgctcgctcgctcactgaggccgcccgggcaaag
cccgggcgtcgggcgacctttggtcgcccggcctcagtgagcgagcgagc
gcgcagagagggagtggccaactccatcactaggggttccttctagacaa
ctttgtatagaaaagttgggaggctgaggggggggaaagggcatgggtgt
ttcatgaggacagagcttccgtttcatgcaatgaaaagagtttggagacg
gatggtggtgactggactatacacttacacacggtagcgatggtacactt
tgtattatgtatattttaccacgatctttttaaagtgtcaaaggcaaatg
gccaaatggttccttgtcctatagctgtagcagccatcggctgttagtga
caaagcccctgagtcaagatgacagcagcccccataactcctaatcggct
ctcccgcgtggagtcatttaggagtagtcgcattagagacaagtccaaca
tctaatcttccaccctggccagggccccagctggcagcgagggtgggaga
ctccgggcagagcagagggcgctgacattggggcccggcctggcttgggt
ccctctggcctttccccaggggccctctttccttggggctttcttgggcc
gccactgctcccgctcctctccccccatcccaccccctcaccccctcgtt
cttcatatccttctctagtgctccctccactttcatccacccttctgcaa
gagtgtgggaccacaaatgagttttcacctggcctggggacacacgtgcc
cccacaggtgctgagtgactttctaggacagtaatctgctttaggctaaa
atgggacttgatcttctgttagccctaatcatcaattagcagagccggtg
aaggtgcagaacctaccgcctttccaggcctcctcccacctctgccacct
ccactctccttcctgggatgtgggggctggcacacgtgtggcccagggca
ttggtgggattgcactgagctgggtcattagcgtaatcctggacaagggc
agacagggcgagcggagggccagctccggggctcaggcaaggctgggggc
ttcccccagacaccccactcctcctctgctggacccccacttcatagggc
acttcgtgttctcaaagggcttccaaatagcatggtggccttggatgccc
agggaagcctcagagttgcttatctccctctagacagaaggggaatctcg
gtcaagagggagaggtcgccctgttcaaggccacccagccagctcatggc
ggtaatgggacaaggctggccagccatcccaccctcagaagggacccggt
ggggcaggtgatctcagaggaggctcacttctgggtctcacattcttgga
tccggttccaggcctcggccctaaatagtctccctgggctttcaagagaa
ccacatgagaaaggaggattcgggctctgagcagtttcaccacccacccc
ccagtctgcaaatcctgacccgtgggtccacctgccccaaaggcggacgc
aggacagtagaagggaacagagaacacataaacacagagagggccacagc
ggctcccacagtcaccgccaccttcctggcggggatgggtggggcgtctg
agtttggttcccagcaaatccctctgagccgcccttgcgggctcgcctca
ggagcaggggagcaagaggtgggaggaggaggtctaagtcccaggcccaa
ttaagagatcaggtagtgtagggtttgggagcttttaaggtgaagaggcc
cgggctgatcccacaggccagtataaagcgccgtgaccctcaggtgatgc
gccagggccggctgccgtcggggacagggctttccatagccactagtgta
agtttagtctttttgtcttttatttcaggtcccggatccggtggtggtgc
aaatcaaagaactgctcctcagtggatgttgcctttacttctagcaagtt
tgtacaaaaaagcaggctgccaccatgagcgccctgagaagaaagttcgg
cgacgactaccaggtggtcaccacaagcagctctggctctggactgcagc
ctcaaggacctggacaggatcctcagcagcagctggtgcccaagaagaaa
cggcagagattcgtggacaagaacggccggtgcaatgtgcagcacggcaa
tctgggcagcgagacaagcagatacctgagcgacctgttcaccacactgg
tggacctgaagtggcggtggaacctgttcatcttcatcctgacctacacc
gtggcctggctgttcatggccagcatgtggtgggttatcgcctacaccag
aggcgacctgaacaaggcccatgtgggcaactacaccccttgcgtggcca
acgtgtacaacttccctagcgccttcctgttcagcatcgagacagaggcc
acaatcggctacggctaccggtacatcaccgacaagtgccccgagggaat
tatcctgttcctgttccagtccatcctgggctctatcgtggatgccttcc
tgatcggctgcatgttcatcaagatgagccagcctaagaagcgggccgag
acactgatgtttagcgagcacgccgtgatcagcatgcgcgacggaaaact
gaccctgatgttcagagtgggcaacctgcggaacagccacatggtgtcag
cccagatcagatgcaagctgctgaagtccagacagacccctgagggcgag
ttcctgcctctggatcagctggaactggacgtgggctttagcacaggcgc
cgatcagctgtttctggtgtcccctctgaccatctgccacgtgatcgacg
ctaagagccccttctacgatctgagccagcggagcatgcagaccgagcag
tttgagatcgtggtcatcctggaaggcattgtggaaaccaccggcatgac
ctgccaggccagaaccagctacacagaggatgaggtgctgtggggccaca
gattcttccccgtgatctctctggaagagggcttcttcaaggtggactac
agccagttccacgccacctttgaggtgcccacacctccttacagcgtgaa
agaacaagaggaaatgctgctgatgagcagccctctgatcgcccctgcca
tcaccaacagcaaagaacggcacaacagcgtggaatgcctggacggcctg
gatgacatcaccaccaagctgccttccaagctgcagaagatcaccggcag
agaggacttccccaagaaactgctgagaatgagcagcaccaccagcgaga
aggcctactctctgggcgacctgcctatgaagctgcagcggatctctagc
gtgcccggcaactctgaggaaaagctggtttccaagaccaccaagatgct
gagcgatcccatgagccagtccgtggctgatctgccacctaaactgcaga
aaatggctggcggagccgccagaatggaaggcaatctgcctgccaaactg
cgcaagatgaactccgaccggttcacctagacccagctttcttgtacaaa
gtgggaattcGATAATCAACCTCTGGATTACAAAATTTGTGAAAGATTGA
CTGGTATTCTTAACTTTGTTGCTCCTTTTACGCTTTGTGGATACGCTGCT
TTATTGCCTTTGTATCTTGCTATTGCTTCCCGTTTGGCTTTCATTTTCTC
CTCCTTGTATAAATCCTGGTTGCTGTCTCTTTTTGAGGAGTTGTGGCCCG
TTGTCAGGCAACGTGGCGTGGTGTGCACTGTGTTTGCTGACGCAACCCCC
ACTGGTTGGGGCATTGCCACCACCTGTCAGCTCCTTTCCGGGACTTTCGC
TTTCCCCCTCCCTATTGCCACGGCGGAACTCATCGCCGCCTGCCTTGCCC
GCTGCTGGACAGGGGCTCGGCTGTTGGGCACTGACAATTCCGTGGTGTTG
TCGGGGAAATCATCGTCCTTTCCTTGGCTGCTCGCCTGTGTTGCCACCTG
GATTCTGCGCGGGACGTCCTTCTGCTACGTCCCTTCGGCCCTCAATCCAG
CGGACCTTCCTTCCCGCGGCCTGCTGCCGGCTCTGCGGCCTCTTCCGCGT
CTTCGCCTTCGCCCTCAGACGAGTCGGATCTCCCTTTGGGCCGCCTCCCC
GCATCGGACTAGctagagctcgctgatcagcctcgactgtgccttctagt
tgccagccatctgttgtttgcccctcccccgtgccttccttgaccctgga
aggtgccactcccactgtcctttcctaataaaatgaggaaattgcatcgc
attgtctgagtaggtgtcattctattctggggggtggggtggggcaggac
agcaagggggaggattgggaagagaatagcaggcatgctggggagggcCg
caggaacccctagtgatggagttggccactccctctctgcgcgctcgctc
gctcactgaggccgggcgaccaaaggtcgcccgacgcccgggctttgccc
gggcggcctcagtgagcgagcgagcgcgcagctgcctgcaggggcgcctg
atgcggtattttctccttacgcatctgtgcggtatttcacaccgcatacg
tcaaagcaaccatagtacgcgccctgtagcggcgcattaagcgcggcggg
tgtggtggttacgcgcagcgtgaccgctacacttgccagcgccttagcgc
ccgctcctttcgctttcttcccttcctttctcgccacgttcgccggcttt
ccccgtcaagctctaaatcgggggctccctttagggttccgatttagtgc
tttacggcacctcgaccccaaaaaacttgatttgggtgatggttcacgta
gtgggccatcgccctgatagacggtttttcgccctttgacgttggagtcc
acgttctttaatagtggactcttgttccaaactggaacaacactcaactc
tatctcgggctattcttttgatttataagggattttgccgatttcggtct
attggttaaaaaatgagctgatttaacaaaaatttaacgcgaattttaac
aaaatattaacgtttacaattttatggtgcactctcagtacaatctgctc
tgatgccgcatagttaagccagccccgacacccgccaacacccgctgacg
cgccctgacgggcttgtctgctcccggcatccgcttacagacaagctgtg
accgtctccgggagctgcatgtgtcagaggttttcaccgtcatcaccgaa
acgcgcgagacgaaagggcctcgtgatacgcctatttttataggttaatg
tcatgataataatggtttcttagacgtcaggtggcacttttcggggaaat
gtgcgcggaacccctatttgtttatttttctaaatacattcaaatatgta
tccgctcatgagacaataaccctgataaatgcttcaataatattgaaaaa
ggaagagtatgagccatattcaacgggaaacgtcttgctctaggccgcga
ttaaattccaacatggatgctgatttatatgggtataaatgggctcgcga
taatgtcgggcaatcaggtgcgacaatctatcgattgtatgggaagcccg
atgcgccagagttgtttctgaaacatggcaaaggtagcgttgccaatgat
gttacagatgagatggtcagactaaactggctgacggaatttatgcctct
tccgaccatcaagcattttatccgtactcctgatgatgcatggttactca
ccactgcgatccccgggaaaacagcattccaggtattagaagaatatcct
gattcaggtgaaaatattgttgatgcgctggcagtgttcctgcgccggtt
gcattcgattcctgtttgtaattgtccttttaacagcgatcgcgtatttc
gtctcgctcaggcgcaatcacgaatgaataacggtttggttgatgcgagt
gattttgatgacgagcgtaatggctggcctgttgaacaagtctggaaaga
aatgcataaacttttgccattctcaccggattcagtcgtcactcatggtg
atttctcacttgataaccttatttttgacgaggggaaattaataggttgt
attgatgttggacgagtcggaatcgcagaccgataccaggatcttgccat
cctatggaactgcctcggtgagttttctccttcattacagaaacggcttt
ttcaaaaatatggtattgataatcctgatatgaataaattgcagtttcat
ttgatgctcgatgagtttttctaactgtcagaccaagtttactcatatat
actttagattgatttaaaacttcatttttaatttaaaaggatctaggtga
agatcctttttgataatctcatgaccaaaatcccttaacgtgagttttcg
ttccactgagcgtcagaccccgtagaaaagatcaaaggatcttcttgaga
tcctttttttctgcgcgtaatctgctgcttgcaaacaaaaaaaccaccgc
taccagcggtggtttgtttgccggatcaagagctaccaactctttttccg
aaggtaactggcttcagcagagcgcagataccaaatactgttcttctagt
gtagccgtagttaggccaccacttcaagaactctgtagcaccgcctacat
acctcgctctgctaatcctgttaccagtggctgctgccagtggcgataag
tcgtgtcttaccgggttggactcaagacgatagttaccggataaggcgca
gcggtcgggctgaacggggggttcgtgcacacagcccagcttggagcgaa
cgacctacaccgaactgagatacctacagcgtgagctatgagaaagcgcc
acgcttcccgaagggagaaaggcggacaggtatccggtaagcggcagggt
cggaacaggagagcgcacgagggagcttccagggggaaacgcctggtatc
tttatagtcctgtcgggtttcgccacctctgacttgagcgtcgatttttg
tgatgctcgtcaggggggcggagcctatggaaaaacgccagcaacgcggc
ctttttacggttcctggccttttgctggccttttgctcacatgt
(SEQ ID NO: 18)

Another object of the present invention is a pharmaceutical composition comprising the vector or the pharmaceutically acceptable carrier of the present invention, with a pharmaceutically acceptable carrier, diluent or excipient.

Another object of the present invention is a vector, a carrier or a pharmaceutical composition of the present invention, for use in treating rod-cone dystrophy (RCD).

Rod-cone dystrophy (RCD) is a heterogeneous group of diseases such as Retinitis Pigmentosa (RP), in particular non-syndromic X-linked Retinitis Pigmentosa (XLRP), autosomal recessive RP, autosomal dominant RP. The most common syndromic forms of RCD include Usher syndrome, Bardet-Biedl syndrome, Refsum disease, Bassen-Kornzweig syndrome and Batten disease.

The RCD subject to be treated is a mammal, in particular a non-human or human primate, preferably a human. The RCD in the mammal may be at an early, intermediate or advanced stage of the disease. In RCD subjects at intermediate or advanced stage of the disease, transduction of the subjects' cones with a nucleotide sequence encoding GIRK1 F137S would be sufficient to achieve vision restoration provided cone opsin and cone arrestin are still expressed in the patients' cone cell bodies. In RCD subjects whose cone cell bodies no longer express cone opsin, transduction of the subjects' cones with a nucleotide sequence encoding GIRK1 F137S and a mammalian cone opsin would be required.

Accordingly, treatment of RCD may be implemented by administering the vector(s), carrier or pharmaceutical composition of the present invention to the mammal, so as to achieve transduction of cones with the GIRK1 F137S transgene, or GIRK1 F137S and mammalian cone opsin transgenes.

In other words, another object of the present invention is a method of treating a RCD in a mammal in need thereof, the method comprising administering to the mammal an effective amount of the vector or the carrier of the pharmaceutical composition of the present invention.

Accordingly, in a first embodiment, the vector comprising a nucleotide sequence encoding GIRK1 F137S, carrier including said vector, or a pharmaceutical composition comprising the vector or carrier is for use in treating rod-cone dystrophy in a RCD mammalian subject whose cone cells still express endogenous cone opsin. According to an embodiment, the vector further comprises a nucleotide sequence encoding a mammalian cone opsin. According to another embodiment, the vector does not comprise a nucleotide sequence encoding a mammalian cone opsin. According to an embodiment, the carrier further includes a vector comprising a nucleotide sequence encoding a mammalian cone opsin. According to another embodiment, the carrier does not include a vector comprising a nucleotide sequence encoding a mammalian cone opsin.

In a second embodiment, the vector comprising a nucleotide sequence encoding GIRK1 F137S, carrier including said vector, or a pharmaceutical composition comprising the vector or carrier is for use in treating rod-cone dystrophy in a RCD mammalian subject whose cone cells no longer express endogenous cone opsin. According to this embodiment, the vector further comprises a nucleotide sequence encoding a mammalian cone opsin, or the carrier further includes a vector comprising a nucleotide sequence encoding a mammalian cone opsin.

Treatment of RCD may also be implemented by transducing a mammalian cone precursor cell with vector(s), carrier or pharmaceutical composition of the present invention, and administering the transduced mammalian cone precursor cell to the retina, in particular to the fovea region, of the RCD mammal.

In other words, another object of the present invention is a method of treating a RCD in a mammal in need thereof, the method comprising administering to the mammal an effective amount mammalian cone precursor cell transduced with the vector or the carrier of the pharmaceutical composition of the present invention.

The invention also relates to a cone precursor cell comprising a heterologous nucleic acid encoding GIRK1 F137S, or encoding GIRK1 F137S and a mammalian cone opsin, for use in a method of treating a RCD. Accordingly, it is also provided a method a method of treating a RCD in a mammal in need thereof, the method comprising administering to the mammal a cone precursor cell comprising a heterologous nucleic acid encoding GIRK1 F137S, or encoding GIRK1 F137S and a mammalian cone opsin. As used herein, the term «heterologous nucleic acid» refers to a gene, polynucleotide or nucleic acid sequence that is not in its natural environment.

Cone precursor cells are not-fully differentiated, non-dividing cells committed to differentiate into cone cells.

In an embodiment, cone precursor cells are obtained from retina of donor (e.g. cadaver eye donor) or from the RCD subject to be treated, preferably from the RCD subject to be treated. In another embodiment, cone precursor cells are obtained from stem cells, in particular embryonic stem cells, induced pluripotent stem (iPS cells), adult stem cells or fetal stem cells. In another embodiment, cone precursor cells are obtained from differentiated embryonic stem cells. According to one embodiment, embryonic stem cells are non-human embryonic stem cells. According to another embodiment, human embryonic stem cells may be used with the proviso that the method itself or any related acts do not include destruction of human embryos. Preferably cone precursor cells are obtained by differentiation of stem cells, preferably from differentiation of adult stem cells or induced pluripotent stem cells, more preferably from differentiation of induced pluripotent stem cells obtained from somatic cells, e.g. fibroblasts, of the RCD subject to be treated.

Embryonic stem cells are able to maintain an undifferentiated state or can be directed to mature along lineages deriving from all three germ layers, ectoderm, endoderm and mesoderm. Embryonic stem cells can be reprogrammed towards cone photoreceptors by manipulation of key developmental signaling pathways as described in the international patent application WO2018055131. For example, it may be used antagonists of the nodal and wnt pathway in addition to activin-A and serum (Watanabe K et al. Nat Neurosci. 2005 March; 8 (3): 288-96.), or inhibition of the Notch signaling pathway can be implemented (Osakada F et al. Nat Protoc. 200; 4 (6): 811-24). Cone precursor cells can be obtained from embryonic stem cells using any protocol known by the skilled person (Osakada F et al. Nat Biotechnol. 2008 February; 26 (2): 215-24; Amirpour N et al. Stem Cells Dev. 2012 January; 21 (I): 42-53; Nakano T et al. Cell Stem Cell. 2012 Jun. 14; 10 (6): 771-85; Zhu Y et al. Plos One. 25 2013; 8 (I): e54552; Yanai A et al. Tissue Eng Part C Methods. 2013 October; 19 (10): 755-64; Kuwahara A et al. Nat Commun. 2015 Feb. 19; 6:6286; Mellough C B et al. Stem Cells. 2015 August; 33 (8): 2416-30; Singh K et al. Stem Cells Dev. 2015 Dec. 1; 24 (23): 2778-95).

Preferably, cone precursor cells are obtained from iPS cells or adult stem cells, more preferably from iPS cells. Induced pluripotent stem (iPS) cells are derived from a non-pluripotent cell, typically an adult somatic cell, by a process known as reprogramming, where the introduction of only a few specific genes are necessary to render the cells pluripotent (e.g. OCT4, SOX2, KLF4 and C-MYC in human cells). One benefit of use of iPS cells is the avoidance of the use of embryonic cells altogether and hence any ethical questions thereof. Photoreceptor precursor cells can be obtained from iPS cells using any differentiation method known by the skilled person.

In particular, photoreceptor precursor cells can be obtained from human iPS cells by a method as disclosed in Garita-Hernandez et al., 2019, Nat Commun 10, 4524. Human iPS are expanded to confluence in IPS medium (e.g. Essential 8™ medium, GIBCO, Life Technologies). After 80% confluence, the medium was switched to a proneural medium (e.g. Essential 6™ medium supplemented with 1% N2 supplement (100×); GIBCO, Life Technologies). The medium was changed every 2-3 days. After 4 weeks of differentiation, neural retina-like structures grew out of the cultures and were mechanically isolated. Pigmented parts, giving rise to RPE were carefully removed. The extended 3D culture in Maturation medium (DMEM/F-12 medium supplemented with 2% B-27™ Supplement (50×), serum free, and 1% MEM Non-Essential Amino Acids Solution (100×); GIBCO, Life Technologies) allowed the formation of retinal organoids. Addition of 10 ng/ml Fibroblast growth factor 2 (FGF2, Preprotech) at this point favoured the growth of retinal organoids and the commitment towards retinal neurons instead of RPE lineage. In order to promote the commitment of retinal progenitors towards photoreceptors, Notch signalling was specifically blocked for a week starting at day 42 of differentiation using the gamma secretase inhibitor DAPT (10 μM, Selleckchem). Floating organoids were cultured in 6 well-plates (10 organoids per well) and medium was changed every 2 days.

Photoreceptor precursor cells can also be obtained from human iPS cells using any other protocol known by the skilled person (Lamba, Osakada and colleagues (Lamba et al. Proc Natl Acad Sci USA. 2006 Aug. 22; 103 (34): 12769-74; Lamba et al. Plos one. 2010 Jan. 20; 5 (l): e8763; Osakada et al. Nat. Protoc. 2009; 4 (6): 811-24; Meyer J S et al. Proc Natl Acad Sci USA. 2009 Sep. 29; 106 (39): 16698-703; Meyer J S et al. Stem Cells. 2011 August; 29 (8): 1206-18; Mellough C B et al. Stem Cells. 2012 April; 30 (4): 673-86; Boucherie C et al. Stem Cells. 2013 February; 31 (2): 408-14; Sridhar A et al. Stem Cells Transl Med. 2013; 2 (4): 255-64; Tucker B A et al. Elife. 2013 Aug. 27,2: e00824; Tucker B A et al. Stem Cells Transl Med. 2013 January; 2 (1): 16-24; eichman S et al. Proc Natl Acad Sci USA. 2014 Jun. 10; 111 (23): 8518-23; Zhong X et al. Nat Commin. 2014 Jun. 10; 5:4047; Wang X et al. Biomaterials. 2015 June; 53:40-9).

The cone precursor comprise a heterologous nucleic acid encoding i) GIRK1 F137S, or ii) encoding GIRK1 F137S and a mammalian cone opsin. Where the cone precursor cells comprise a heterologous nucleic acid encoding GIRK1 F137S, or a functional derivative thereof, and a mammalian cone opsin, the cone precursor cells either comprise i) a heterologous nucleic acid encoding both GIRK1 F137S and a mammalian cone opsin, or ii) a heterologous nucleic acid encoding GIRK1, and another heterologous nucleic acid encoding a mammalian cone opsin.

Said cone precursor cells may be prepared by introducing into said cone precursor cells said heterologous nucleic acid(s), or an expression cassette or vector comprising said nucleic acid(s), by any method known to the skilled person. According to an embodiment, a cone precursor cell comprising a heterologous nucleic acid encoding GIRK1 F137Sf, or encoding GIRK1 F137Sand a mammalian cone opsin, is prepared by infecting the cone precursor cell with a viral vector as described above, in particular with an AAV vector, preferably the AAV8, AAV2-7m8 or AAV9-7m8.

In another aspect, the invention therefore further refers to a method of preparing a cone precursor cell comprising a heterologous nucleic acid encoding GIRK1 F137S, or encoding GIRK1 F137Sand a mammalian cone opsin, said method comprising infecting cone precursor cells with a viral vector or carrier according to the invention, and recovering infected cone precursor cells.

The vector, carrier, or pharmaceutical composition, or cone precursor cells may be administered by any suitable route known to the skilled person in particular by intravitreal or subretinal or suprachoroidal administration.

In an embodiment, the viral vector, carrier or pharmaceutical composition is injected at a dose comprised between 10⁷ to 10¹⁵ vg/eye, preferably between 10¹¹ and 10¹⁵ vg/eye, even more preferably 10¹¹ and 10¹³ vg/eye.

In an embodiment, the viral vector, carrier or pharmaceutical composition is administered by intravitreal injection.

In one embodiment, the AAV vector is an AAV2 or AAV9 or modified versions thereof such as AAV2-7m8 or AAV9-7m8 and said vector is administered by intravitreal injection.

In an embodiment, the viral vector, carrier or pharmaceutical composition is administered by subretinal injection.

The fovea is a small region in the central retina of primates of approximately equal to or less than 0.5 mm in diameter that contains only cone photoreceptor cells, and highest density of cones in the whole retina. The fovea dominates the visual perception of primates by providing high-acuity color vision. The highest density of cones is found at the center of the fovea (<0.3 mm from the foveal center), devoid of rod photoreceptors. Cone density decreases by up to 100-fold with distance from the fovea.

Cone cells in the fovea are the primary targets of gene therapies aiming to treat inherited retinal diseases like retinitis pigmentosa. Usually, viral vectors encoding therapeutic proteins are injected “subretinally”, i.e. into the subretinal space between the photoreceptors and the retinal pigment epithelium (RPE) cells in order to provide gene delivery to cones.

The subretinal delivery leads to the formation of a “bleb”, which refers to a fluid-filled pocket within the subretinal space of the injected eye. In this approach, gene delivery is limited to cells that contact the local bleb of injected fluid. Retinal detachment, and in particular foveal detachment, that occurs during subretinal injections is a concern in eyes with retinal degeneration.

Advantageously, when the vector is an AAV9-7m8 vector (in particular AAV9-7m8-pR1.7 vector), the vector (or carrier of pharmaceutical composition comprising said vector) can be administered by a distal subretinal injection, or in the periphery of the fovea, and then spread laterally to reach the foveal region. According to an embodiment the bleb is formed greater than or equal to 0.5 millimeters away from the center of the fovea, without detaching the foveal region. In an embodiment, said AAV9-7m8 viral vector is formulated in a solution and 50 to 100 μL of solution are injected continuously in 20 to 30 seconds. In an embodiment, said AAV9-7m8 viral vector is formulated in a solution at a concentration of 1×10¹⁰ to 1×10¹² vg/mL (viral genome/mL), preferably of 0.5×10¹¹ to 5×10¹¹ vg/mL, still preferably of 1×10¹¹ vg/mL. Preferably, the cone precursor cells are administered by intraocular injection, preferably by subretinal space injection, more preferably by injection between the neural retina and the overlying PE. The amount of cone precursor cells to be administered may be determined by standard procedure well known by those of ordinary skill in the art. Physiological data of the patient (e.g. age, size, and weight) and type and severity of the disease being treated have to be taken into account to determine the appropriate dosage. The cone precursor cells may be administered as a single dose or in multiple doses. In particular, each unit dosage may contain, from 100,000 to 300,000 cone precursor cells per μl, preferably from 200,000 to 300,000 cone precursor cells per μl.

### BRIEF DESCRIPTION OF THE FIGURES

FIG. 1 represents phototransduction cascade (A) normal phototransduction cascade (B) short phototransduction cascade with an animal opsin and GIRK2 channel. PDE: phosphodiesterase. CNG: cyclic-nucleotic gated channels. cGMP: cyclic guanosine monophosphate.

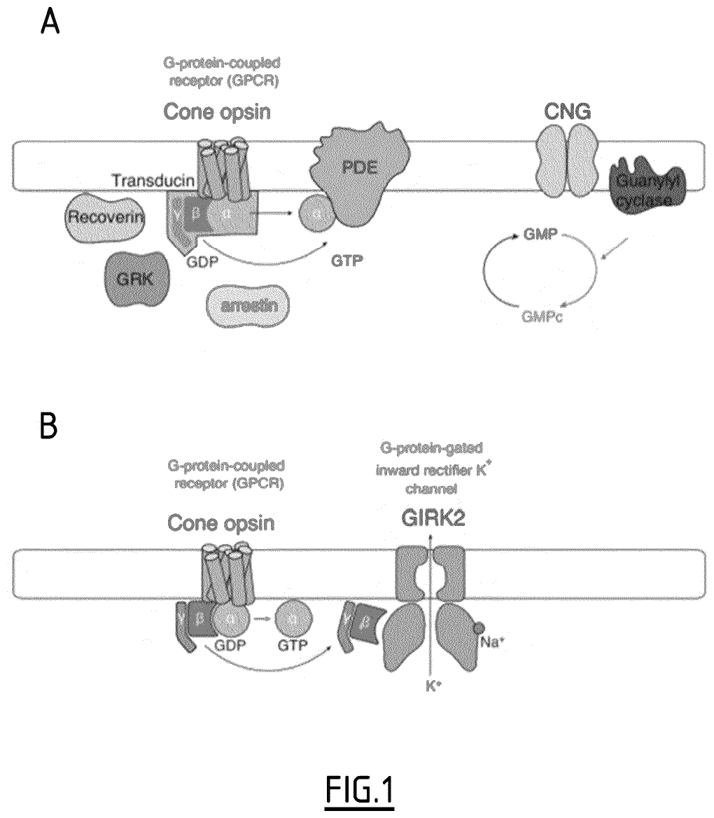

FIG. 2 represents plasmids (A) CMV-GIRK2-GFP and (B) CMV-SWO-mCherry.

FIG. 3 represents what remained in the phototransduction cascade in rd10 mice using immunohistochemistry (A-D) retinal cross-section of a control WT mouse stained with (A) opsin, (B) transducin, (C) PDE and (D) cone arrestin. (E-H) retinal cross-section of a rd10 mouse at P14 stained with (E) opsin, (F) transducin, (G) PDE and (H) cone arrestin. (I-L) Retinal cross-section of a rd10 mouse at P150 stained with (I) opsin, (J) transducin, (K) PDE and (L) cone arrestin. ONL: outer nuclear layer. INL: inner nuclear layer. GC: ganglion cells. Scale bar is 50 μm. Inset scale bar is 25 μm.

FIG. 4 represents preliminary data. (A) Eye fundus of GIRK2-GFP expression in rd10 mouse one week post-injection (*site of injection) (B) Photopic ERG amplitude in rd10 mice at P33, injected with AAV-SWO-tdTomato and AAV-GIRK2-GFP. Control mice are injected with AAV-GFP (n=12). P=0,0002. (C) Representative flickers ERG at P33. (D) Measure of the visual acuity by optokinetic test in rd10 mice, injected with AAV-SWO-tdTomato and AAV-GIRK2-GFP. Control mice are injected with AAV-GFP. Control mice were injected with AAV-GFP (n=8).

FIG. 5 represents GIRK2-mediated vision. (A) Photopic ERG amplitude in rd10 mice at P41, injected with AAV-SWO-tdTomato and/or AAV-GIRK2-GFP. Control mice are injected with AAV-GFP (n=12). P_{SWO+GIRK2}=0,0381 and P_{GIBK2}=0,0021. (B) Measure of the visual acuity by optokinetic test in rd10 mice, injected with AAV-SWO-tdTomato and/or AAV-GIRK2-GFP. Control mice are injected with AAV-GFP. Control mice were injected with AAV-GFP (n=7). (C) Representative flickers ERG at P41.

FIG. 6 represents long term efficiency. (A) Photopic ERG amplitude in rd10 mice, injected with AAV-GIRK2-GFP. Control mice are injected with PBS (n=6). (B) Measure of the visual acuity by optokinetic test in rd10 mice, injected with AAV-GIRK2-GFP. Control mice are injected with PBS (n=6). (C) Number of cones of wild-type mice and non-injected rd10 mice over time (n=6). Pvalue_{(P50-P365)}=0.0022. (D) Linear regression correlation between the ERG amplitudes and the number of cones in rd10 mice (n=6). Pvalue_{non-injected}=0,0482. Pvalue_{AAV-GIRK2-GFP}=0,0007. Pvalue_{PBS}=0,0104.

FIG. 7 represents what remained in the phototransduction cascade in huP347S^{+/-} mice using immunohistochemistry. (A-D) Retinal cross-section of a control WT mouse stained with (A) opsin, (B) transducin, (C) PDE and (D) cone arrestin. (E-H) retinal cross-section of a huP347S^{+/-} mouse at P14 stained with (E) opsin, (F) transducin, (G) PDE and (H) cone arrestin. (I-L) retinal cross-section of a huP347S″ mouse at P150 stained with (I) opsin, (J) transducin, (K) PDE and (L) cone arrestin. ONL: outer nuclear layer. INL: inner nuclear layer. GC: ganglion cells. Scale bar is 50 μm. Inset scale bar is 25 μm.

FIG. 8 represents universality of the approach. (A) Photopic ERG amplitude in huP347S^{+/-} mice, injected with AAV-GIRK2-GFP. Control mice are injected with PBS (n=6). (B) Measure of the visual acuity by optokinetic test in huP347S^{+/-} mice, injected with AAV-GIRK2-GFP. Control mice are injected with PBS (n=6). (C) Number of cones of wild-type mice and non-injected huP347S^{+/-} mice over time (n=6). Pvalue_{(P50-P365)}=0,0022. (D) Linear regression correlation between the ERG amplitudes and the number of cones in huP347S^{+/-} mice (n=5). Pvalue_{non-injected}=0,0313. Pvalue_{AAV-GIRK2-GFP}=0,0146. Pvalue_{PBS}=0,0497.

FIG. 9 represents the efficiency of the mouse GIRK2 in HEK cells transfected with two plasmids: CMV-SWO-mCherry and CMV-GIRK2-GFP.

FIG. 10 represents phenotyping of a normal volunteer and retinitis pigmentosa patients for eligible patient population. Upper panel (A) shows the fundus and OCT images of the back of the eye in a normal individual along with adaptive optics images of cone dominated regions of the retina. Middle panel (B) shows a pie-chart distribution of advanced RCD patients. Lower panels represent OCT and AOSLO images of different patients. AOSLO confocal (up) and AOSLO split detection (low) in vivo retinal images of a patient with retinitis pigmentosa (age 77, male). Acquired fields are located at the transition between regions of presumed dormant cones (i.e. morphologically intact—as indicated by the IS/OS line visible in OCT, clear inner segment mosaic visible in AOSLO split detection, and clear cone mosaic indicating intact inner and outer segments in AOSLO confocal-though with reduced function according to the patient's visual acuity; yellow bars) and damaged or absent cones (indicated by the absent IS/OS line in OCT, and the blurred inner segment and cone mosaics in AOSLO split detection and confocal respectively; red bars). Yellow arrows indicate cones that appear to be degenerating, with absent OS in confocal but present IS in split detection. Scale bars, 200 μm.

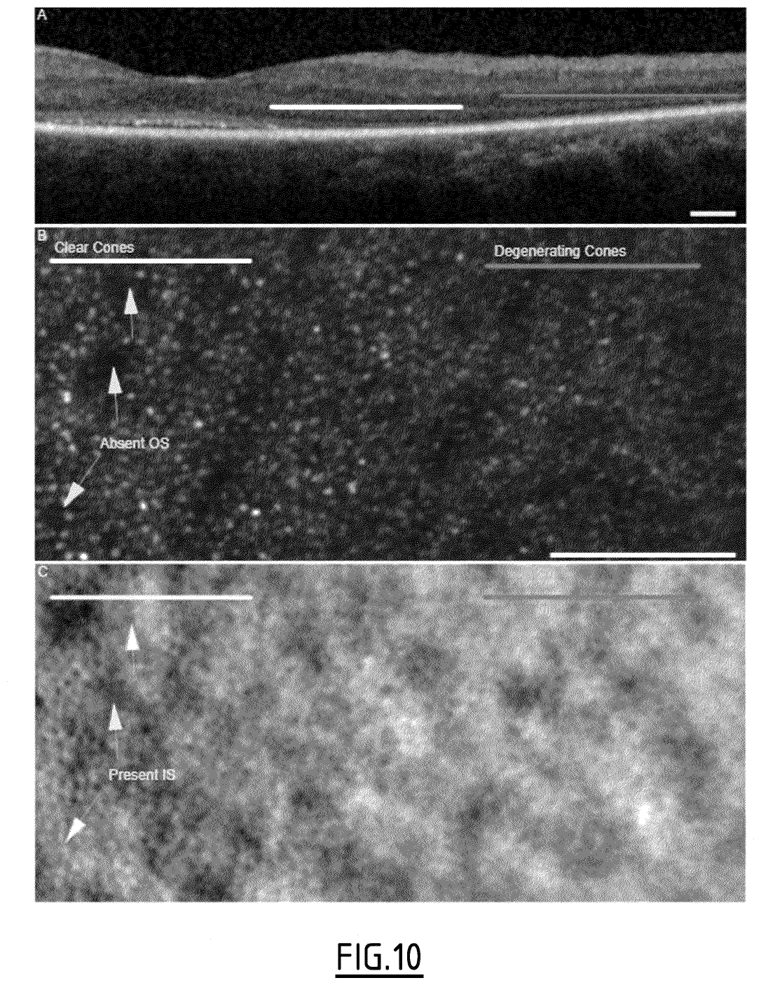

FIG. 11 represents immunohistochemistry labeling cone phototransduction cascade proteins in normal and RP human retina. (A) Retinal cross-section of a 86 years old control human retina (20×). (B) Retinal cross-section of a 75 years old human retina affected by retinitis pigmentosa (RP) and having night blindness and loss of peripheral vision (40×). (A-B) stained with Opn1mw, (bright) and nuclear stain DAPI (dark). ONL: outer nuclear layer. INL: inner nuclear layer. GC: ganglion cells. Scale bar is 50 μm. Inset scale bar is 25 μm.

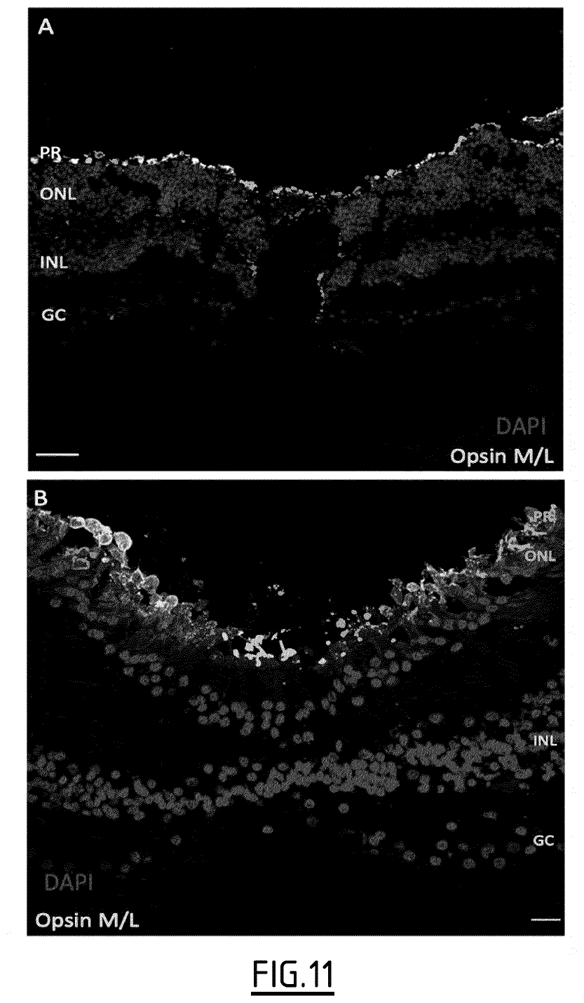

FIG. 12 shows characterization of GIRK1 F137S currents elicited by activation of mOpn4L in HEK293 cells. A) When activated with blue light (471 nm, exposure from 10 to 20 s on the axis of abscissae) mOpn4L induces a strong GIRK1 F137S-mediated current that concludes upon stimulation with lime light (560 nm, exposure from 20 to 60 s on the axis of abscissae) and results in a statistically significant difference in B) the flux of ions through HEK293 membranes and C) the electrical current density per capacitance.

FIG. 13 shows characterization of GIRK1 or GIRK2 currents elicited by activation of mOpn4L in HEK293 cells when activated with blue light (471 nm, exposure from 10 to 20 s on the axis of abscissae) and terminated upon stimulation with lime light (560 nm, exposure from 20 to 60 s on the axis of abscissae).

FIG. 14: shows the current density per capacitance of HEK293 cells expressing A1) hGIRK1 F137S, B1) hGIRK2 or C1) truncated rGIRK2. The current response, elicited by the activation of mOpn4, is shown in comparison to constructs with c-terminal eGFP fusion and untransfected cells. The current density of individual cells is illustrated by individual data points. Significant differences between conditions are marked with * (Kruskal-Wallis One Way Analysis of Variance on Ranks with all pairwise multiple comparison procedures (Dunn's Method) P<0.05). Significant differences between conditions are marked with * (Mann-Whitney Rank Sum Test; P=0.002). When activated with blue light mOpn4 induces a GIRK-mediated current that concludes upon stimulation with lime light as visible in exemplary traces of A2) hGIRK1 F137S, B2) hGIRK2 and C2) truncated rGIRK2 T constructs.

FIG. 15A) Stimulation of HEK293 cells expressing mOpn4 and either hGIRK1 F137S, hGIRK1 F137S-eGFP, or hGIRK2 produces a reliably observable current in whole-cell patch clamp recordings, while currents of hGIRK2-eGFP, rGIRK2 or rGIRK2-eGFP after mOpn4 activation are less likely to be observable. B) Current density per capacitance of HEK293 cells expressing the different GIRK constructs (top) or their eGFP tagged versions (bottom). The current density of individual cells is illustrated by individual data points. Significant differences between conditions are marked with * (Kruskal-Wallis One Way Analysis of Variance on Ranks with all pairwise multiple comparison procedures (Dunn's Method) P<0.05).

FIG. 16 represents the visual acuity in rd10 mice after bilateral subretinal injection at P15 of AAV8-pR1.7-hGIRK1F137S at 5E7 or 5E8 vg/eye compared to vehicle-injected rd10 mice or naïve (not injected) rd10 mice.

FIG. 17 represents the plasmid pAAV2-5′ITR-pR1.7-hGIRK-F137S-BGHpA-AAV2-3′ITR.

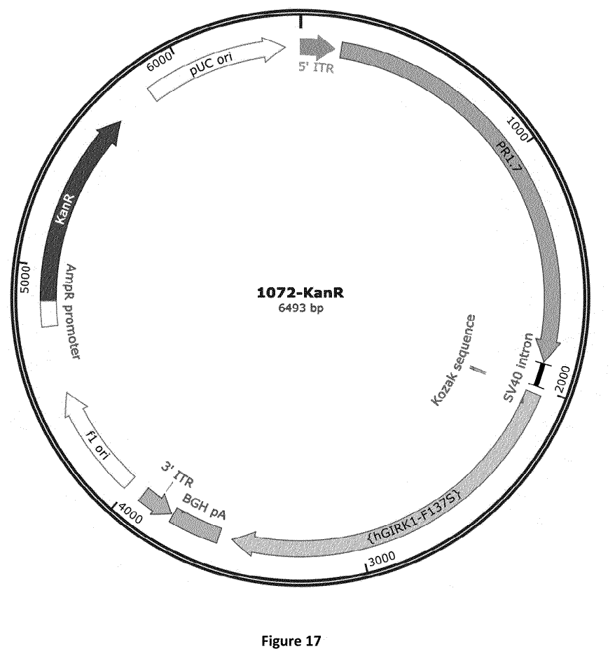

FIG. 18 represents the plasmid pAAV2-5′ITR-pR1.7-hGIRK-F137S-WPREmut6-BGHpA-AAV2-3′ITR.

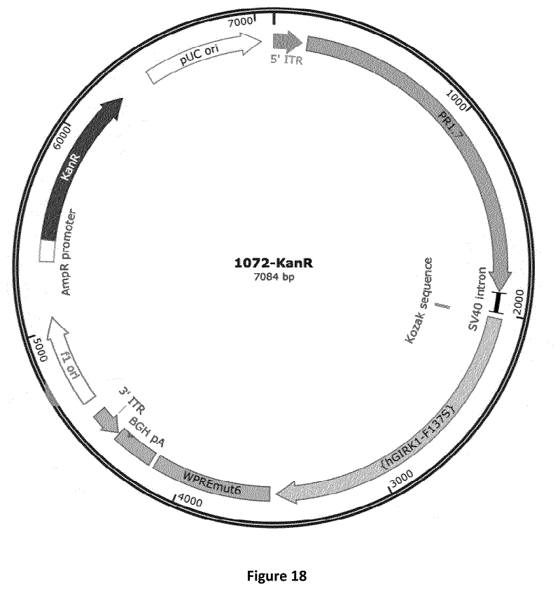

FIG. 19 represents the plasmid pAAV2-5′ITR-pR1.7-hGIRK-F137S-WPREmut6deltaATG-BGHpA-AAV2-3′ITR.

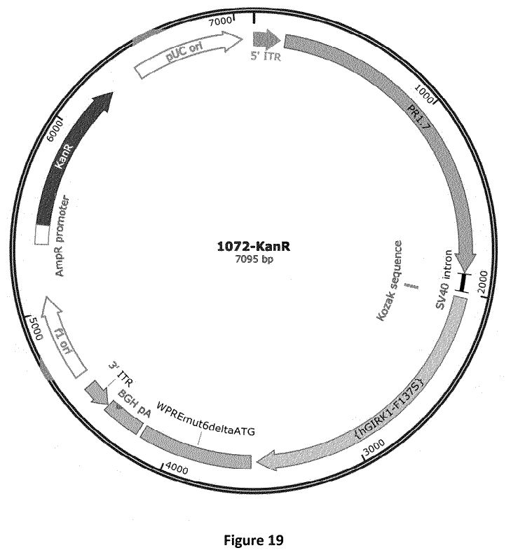

### EXAMPLES

### Example 1: Material and Methods

### 1. Animals

C57BL/6j^{rd10/rd10} (rd10) mice were used in these experiments. They have a mutation on the rod PDE gene leading to a dysfunctional phototransduction cascade and a rod-cone dystrophy. The second model used is the huRhoP347S^{+/-} mouse. The homozygous strand of this mouse present a KO of mouse rhodopsin (mRho) gene and a KI of human rhodopsin (huRho) with a mutation (P347S) (Millington-Ward et al., 2011) [30]. The homozygous males were crossed C57BL/6j (wild-type) females to obtain heterozygous mice. These mice have a similar phenotype as the rd10 mice but the degeneration rate is lower.

### 2. AAV Injections

Mice were first anesthetised with intraperitoneal injections of 0.2 ml/20 g ketamine (Ketamine 500, Vibrac France) and xylazine (Xylazine 2%, Rompun) diluted in 0.9% NaCl. Eyes were dilated with 8% Neosynephrine (Neosynephrine Faure 10%, Europhta) and 42% Mydriaticum (Mydriaticum 0.5%, Thea) diluted in 0.9% NaCl.

A total volume of 1 μl of vector solution was injected subretinally. Fradexam, an ophthalmic ointment, was applied after injection. The list of injected viral vectors is presented below:

Injection Table

| | | viral vector | viral vector | volume |
|---|---|---|---|---|
| Mice | Eyes | injected | titration | injected |
| rd10 | both | AAV8-mCAR-GFP | 10¹¹ | 1 μl for all |
| rd10 | both | AAV8-mCAR-GIRK2-GFP + | rAAV particles | conditions |
| | | AAV8-mCAR-SWO- | | |
| | | tdTomato | | |
| rd10 | both | AAV8-mCAR-GIRK2-GFP | | |
| rd10 | right | PBS | | |
| rd10 | left | AAV8-PR1.7-GIRK2-GFP | 5.10⁸ rAAV | |
| | | | particles | |
| huRhoP347S^{+/-} | right | PBS | | |
| huRhoP347S^{+/-} | left | AAV8-PR1.7-GIRK2-GFP | 5.10⁸ rAAV | |
| | | | particles | |

### 3. Eye Fundus Examination

One week after subretinal injection, mice were anesthetised by isofluorane inhalation. Eyes were dilated and then protected with Lubrithal eye gel (VetXX). Fundus imaging was performed with a fundus camera (Micron III; Phoenix research Lab) equipped with specific filters to monitor GFP or tdTomato expression in live anesthetised mice.

### 4. Electroretinography (ERG) Recordings

To evaluate retinal function, electroretinography recordings (ERG) were recorded (espion E2 ERG system; Diagnosys). Several tests were performed at different time points after injections of the viral vectors. Mice were anesthetised with intraperitoneal injections of 0.2 ml/20 g ketamine (Ketamine 500, Vibrac France) and xylazine (Xylasine 2%, Rompun) diluted in 0.9% NaCl. Mice were then placed on a heated pad at 37° C. Eyes were dilated with Neosyhephrine (Neosynephrine Faure 10%, Europhta) and Mydriaticum (Mydriaticum 0.5%, Thea) diluted in 0.9% NaCl. Eyes were protected with Lubrithal eye gel before putting electrodes on the corneal surface of each eye. The reference electrode was inserted under the skin into the forehead and a ground electrode under the skin in the back.

ERG recordings were done under two conditions: (i) photopic condition, which reflects con-driven light responses—6 ms light flashes were applied every second during 60 seconds at increasing light intensities (0.1/1/10/50cd s/m) after an adaptation of 5 minutes at 20 cd s/m and (ii) flicker condition, which are rapid frequency light stimuli that reflect cone function (70 flashes at 10 Hz et 1 cd s/m).

Graph and statistical analysis were performed using GraphPad.

### 5. Optokinetic Test

Visual acuity was measured using an optokinetic test scoring the head turning movement of a mouse placed in front of moving bars. Testing was performed using a computer-based machine consisting of four computer monitors arranged in a square to form an optokinetic chamber. A computer program was designated to generate the optokinetic stimuli, consisting of moving alternate black and white stripes. The spatial frequency is ranging from 0.03 to 0.6 cyc/deg. The program enabled modulation of stripe width and direction of bar movement.

### 6. Immunohistochemistry and Confocal Imaging

Animals were sacrificed by CO₂ inhalation, and the eyes were enucleated and fixed in 4% paraformaldehyde-PBS for 1 h at room temperature. The eyes were dissected either as eyecups for immunohistochemistry or prepared as flat mounts for cell counting. The eyecups were then cryoprotected with a gradient of PBS-Sucrose 10% for 1 h and then in PBS-Sucrose 30% overnight. The eyecups were embedded in OCT and 12 μm thick cryostat sections (ThermoFisher) were cut and mounted on glass slides. The sections were washed in PBS (3×5 mins) and stained against different antibodies (see table below) and DAPI (1:2000). The sections were finally washed in PBS, mounted in Fluoromount Vactashield (Vector Laboratories) and coverslipped for imaging using laser-confocal microscopy (Olympus IX81). For flat-mount retina stainings, the protocol is the same except that the tissue was not cryoprotected. Images were analysed using FIJI software.

Antibody table

| | | |
|---|---|---|
| Target | Host | Clonality/Conjugated |

PRIMARY ANTIBODIES

| | | |
|---|---|---|
| Red/Green opsin (M/L | Rabbit | Polyclonal |
| opsin) | | |
| Mouse Cone arrestin | Rabbit | Polyclonal |
| PDE6C | Rabbit | Polyclonal |
| GNAT2 | Rabbit | Polyclonal |

| | |
|---|---|
| PNA-Lectin | Conjugated with FITC |

SECONDARY ANTIBODIES

| | | |
|---|---|---|
| Anti-rabbit | Donkey | AF 546 |

### 7. Cell Counts

Flat mount retinas of rd10 and huRhoP347S^{+/-} mice were stained using antibodies against mouse cone arrestin—mCAR (1:10000) and DAPI (1:2000). The double stained cells counted at different ages. Retinas from 5 animals (n=10) were used for each age and were oriented dorso-ventrally and naso-temporally. Serial optical sections were obtained to cover the thickness of the entire outer nuclear layer (ONL). Two scanning areas of 211.97×211.97 μm were made in each of the four regions in all retinas. Counts of cone cells were performed manually using the FIJI software by the reconstruction of the images (z stack) covering the entire thickness of the ONL. Average density values of each retina were calculated to obtain the number of cone cells per mm² at different ages.

### 8. In Vitro Test of the Efficiency of Mouse GIRK2

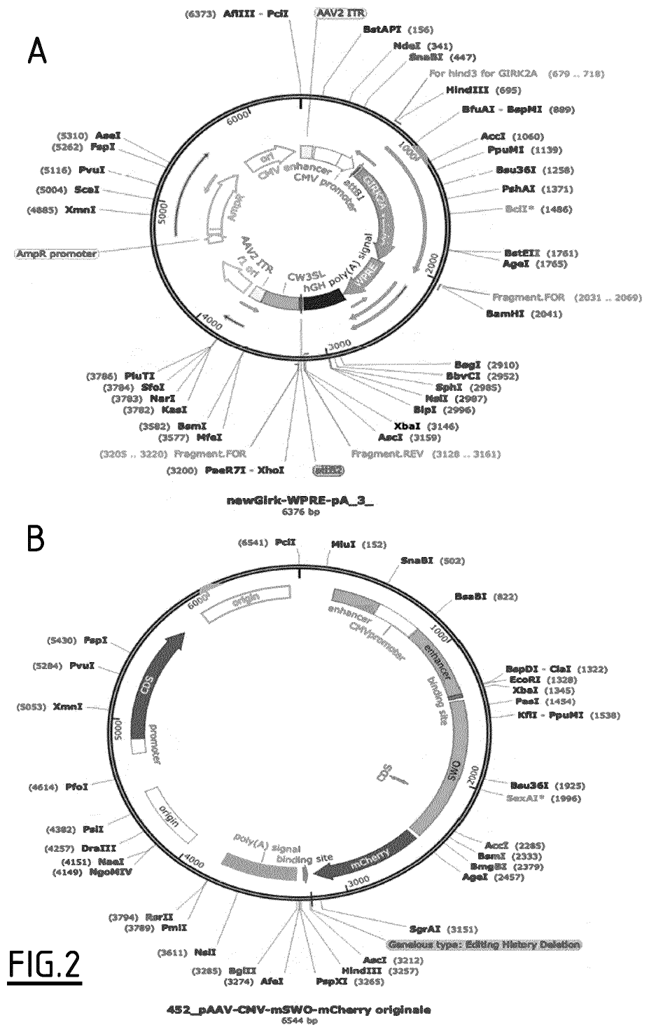

HEK cells were transfected with two plasmids: CMV-SWO-mCherry and CMV-GIRK2-GFP (FIG. 2) according to a well-known procedure in the art. HEK293 cells were cultured and recorded in dark room conditions after transfection. Cells were placed in the recording chamber of a microscope equipped with a 25× water immersion objective (XLPlanN-25×-W-MP/NA1.05, Olympus) at 36° C. in oxygenated (95% O2/5% CO2) Ames medium (Sigma-Aldrich) enriched with an addition of 1 mM9-cis-retinal. KGluconate was added to the external solution in order to get a high extracellular potassium concentration leading to a cell potassium reversal potential of −40 mV.

For Whole-cell recordings, the Axon Multiclamp 700B amplifier (Molecular Device Cellular Neurosciences) was used, GIRK-mediated K+-currents were recorded in voltage-clamp configuration at −80 mV, using borosilicate glass pipettes (BF100-50-10, Sutter Instrument) pulled to 5MΩ and filled with 115 mMK Gluconate, 10 mM KCl, 1 mM MgCl2, 0.5 mM CaCl2, 1.5 mM EGTA, 10 mM HEPES, and 4 mM ATP-Na2 (pH 7.2).

During experiments, a CCD camera (Hamamatsu Corp.) was used to visualize cells using a trans-illuminated infrared-light. A monochromatic light source (Polychrome V, TILL photonics) was used to stimulate cells during electrophysiological experiments with light flashes at 400 nm.

### 9. Patient Eye Fundus Imaging

Adaptive optics scanning laser ophthalmoscopy (AOSLO) (Roorda et al Opt Exp 2002) was used to image cone photoreceptor mosaic at cell resolution. The AOSLO device used (MAORI, PSI, Andover, MA, USA) allows simultaneous imaging over a 2-degree field of view of intact cones with both inner and outer segments (IS, OS) from light scattered along the optical axis (confocal mode) and inner segments (IS) from multiply scattered light scattered off axis (split detection mode). This allows us to evaluate cone presence and health, with differential imaging of IS versus IS+OS for each cone.

### Example 2: Results

### 1. The Changes in the Phototransduction Cascade in Degenerating Cones

The phototransduction cascade was first analysed in the rd10 mouse model by studying its components using immunohistochemistry, at different time points during retinal degeneration. Immunofluorescence staining was performed against cone opsin, transducing, phosphodiesterase and cone arrestin proteins of the phototransduction cascade that interact directly with cone opsin.

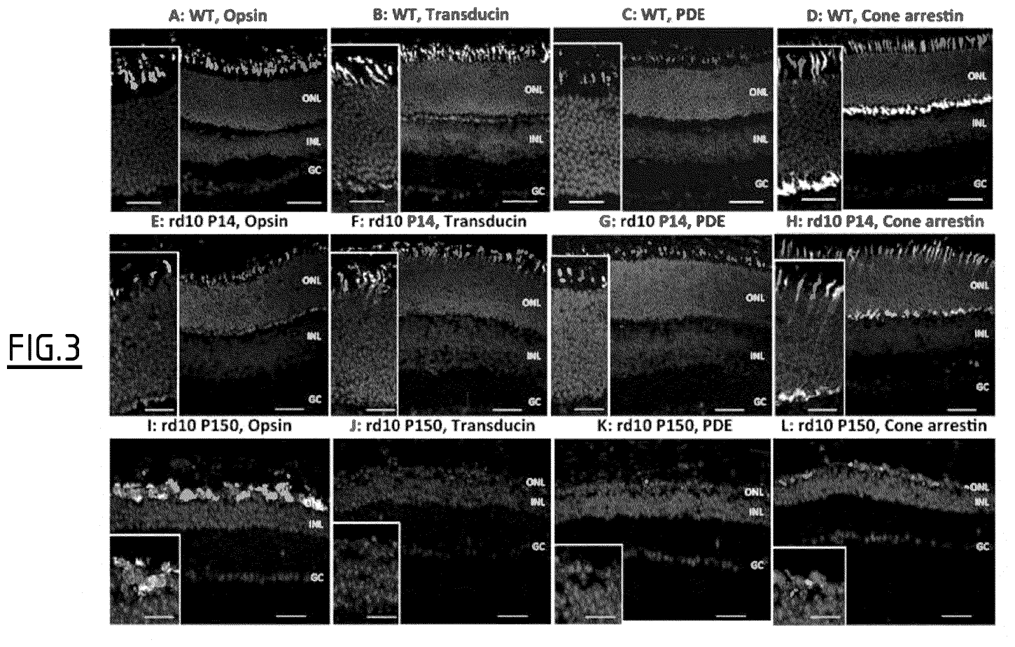

FIG. 3 shows that only the cone opsin and arrestin were still expressed and localized around the cone cell body at late stage of the disease.

### 2. Cone Opsin and GIRK2-Mediated Vision Restoration

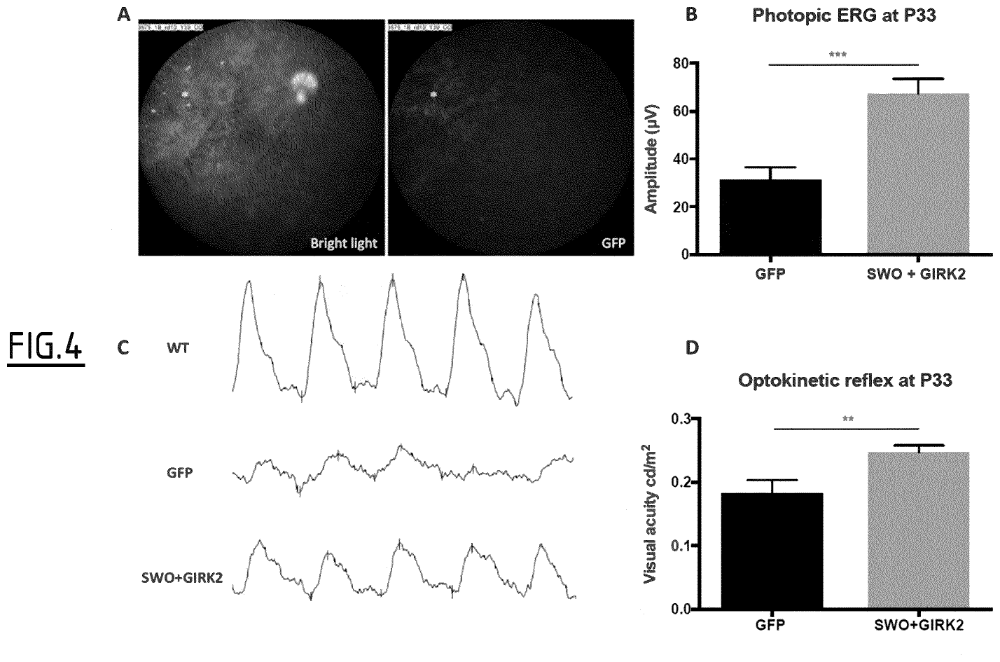

Based on immunohistochemistry and previous findings with cone opsins expressed in neurons, it was first studied why delivering a mouse short wavelength cone opsin (SWO) fused with tdTomato and rat truncated GIRK2 fused with GFP using two AAV vectors mixed in equimolar ratios would enhance cone cell's response to light. Thus two AAVs were injected subretinally to degenerating rd10 mouse retinas at p15 (FIG. 4A). This led to a significant increase in phototpic ERG amplitudes in treated eyes compared to controls (FIG. 4B). Flicker ERGs confirmed that the recovery mechanism was still active in these cone cells expressing GIRK2 allowing them to follow a fast stimulus (FIG. 4C). The rd10 animals treated with GIRK2 showed also an improved optokinetic reflex compared to controls (FIG. 4D).

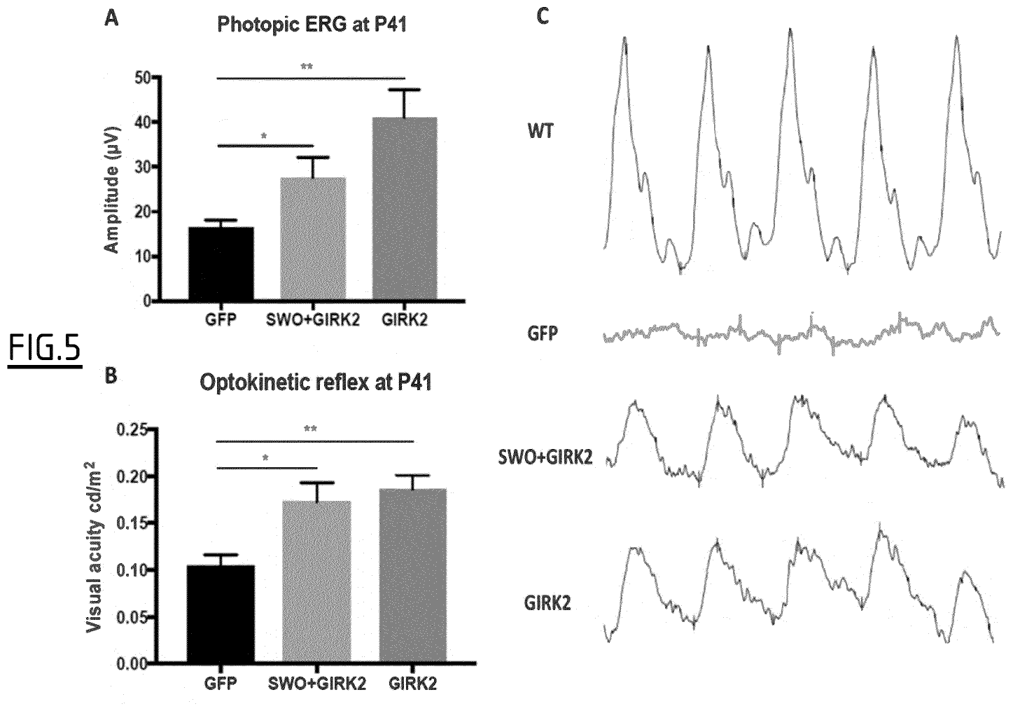

Next it was studied if the endogenous cone opsin, still present in degenerating cones, was functional and sufficient to activate GIRK2 channel in this mouse model. For this, a single AAV8 vector encoding GIRK2 in fusion with GFP was delivered. This led to similar increases in photopic ERG amplitudes and optokinetic reflex in treated eyes compared to controls confirming that GIRK2 alone was sufficient to increase light sensitivity via G protein coupled signalling involving cone opsin (FIG. 5A-B). Flicker ERGs were also robustly amplified with this approach (FIG. 5C).

### 3. GIRK2-Mediated Vision Restoration: Long-Term Efficacy

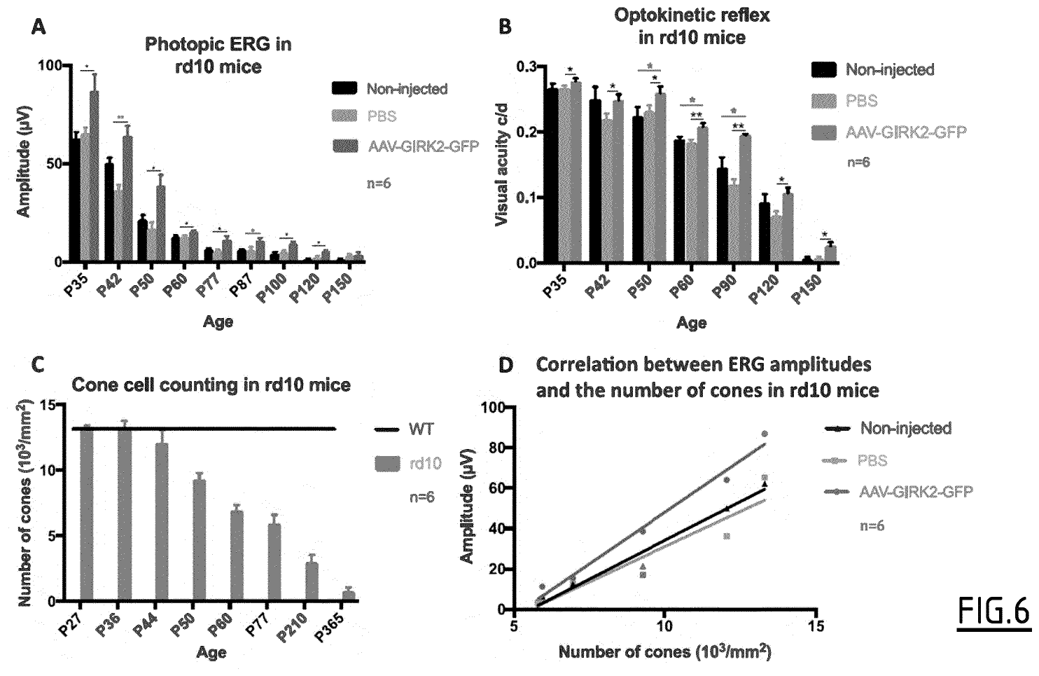

Photopic ERG recordings were performed to monitor the cone response to light stimuli at different time points after treatment with GIRK2 and in absence of treatment. These ERGs were done under two conditions: (i) photopic with light flashes applied every second during 60 seconds at increasing light intensities and (ii) flicker stimulation with repetitive flashes during 60 seconds. Data were collected on a weekly basis until p50 and then every 10 to 13 days until 11 weeks of age and showed a gradual decline in ERG amplitudes for both controls and treated eyes (FIG. 6A). Moreover, these results are consistent with the optokinetic test, both controls and treated eyes with GIRK2 show a decreased optokinetic reflex over time (FIG. 6B). This decline was to be expected as cone numbers also decreased over time in the rd10 mice (FIG. 6C). The number of cone photoreceptors remaining in rd10 retinas were counted to correlate decreases in cone numbers with decrease in ERG amplitudes. Indeed, the decrease in light responses was proportional to number of remaining photoreceptors (FIG. 6D). It was thus concluded that GIRK2 increased light responses in remaining cones so long as cones remained alive but, as expected, it did not slow down the loss of cone cells.

### 4. GIRK2-Mediated Vision Restoration in an RCD Model Caused by Mutant Rhodopsin

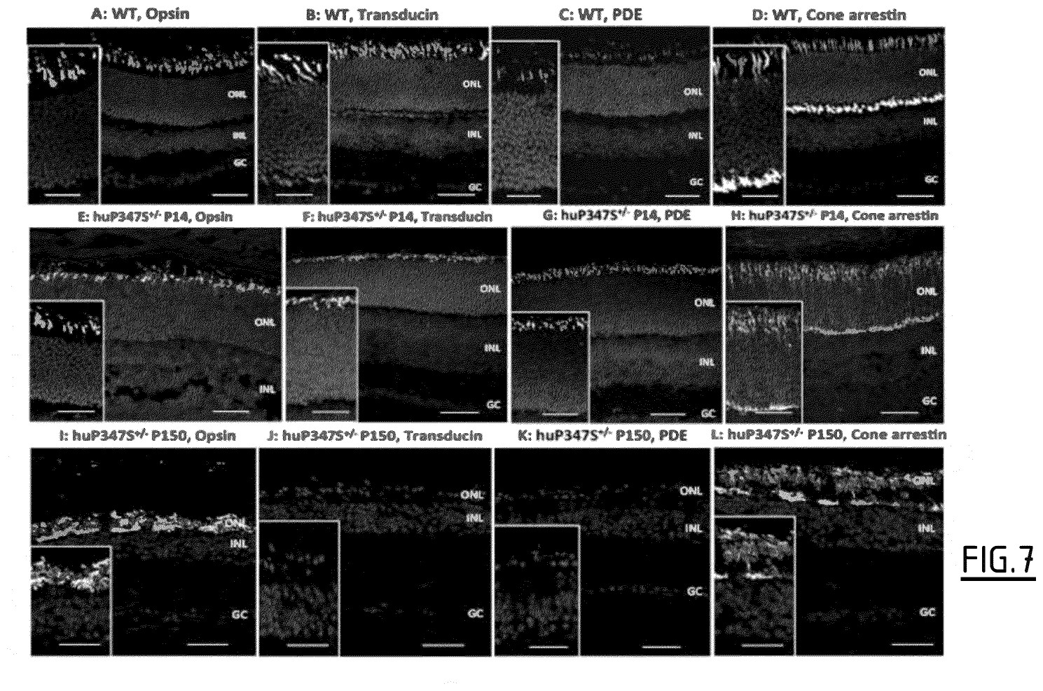

Having in mind the goal of creating a mutation-independent therapy, the approach was tested in another mouse model—with a different causal mutation. To this aim experiments were done in a heterozygous mouse model called mRho^{-/-}huRhoP347S^{+/-} carrying a knock in for P347S mutant human rhodopsin. Mutant human rhodopsin and absence of mouse rhodopsin led to a rod-cone dystrophy in this complementary model. Here, the same set of experiments was repeated as that was done in the rd10 mouse model. First, the phototransduction cascade proteins interacting with cone opsin at different time points was analysed (FIG. 7). It was noticed that: (i) the degeneration rate was slower compared to rd10 and (ii) similar to the rd10 model only the opsin and the arrestin persisted in cone cell bodies at P150.

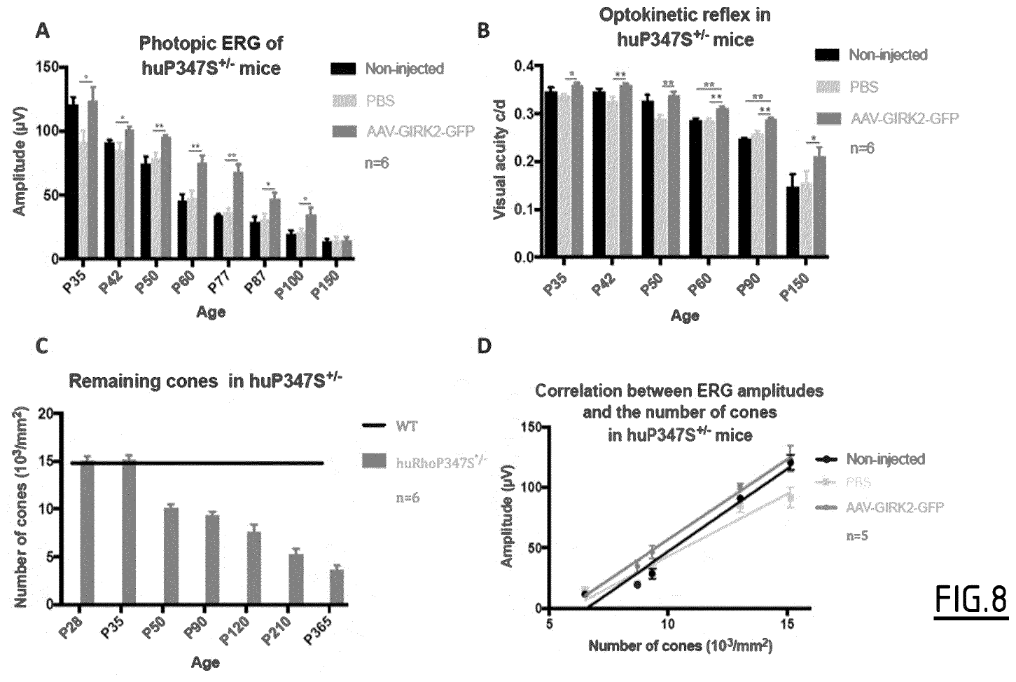

Next, mice were injected at P15 with the same AAV vectors encoding for GIRK2 fused with GFP and recorded ERGs to monitor cone response to light stimuli at various time points (FIG. 8A). The response amplitudes of treated eyes were significantly higher than that of control eyes until P100. Moreover, flicker ERG responses were also similarly improved in this mouse model. Similarly to rd10 mice, this mouse model also shows an improved optokinetic reflex that decreases over time in both control and treated conditions (FIG. 8B). This decline is to be expected as cone numbers also decreases over time in this RCD mouse model (FIG. 8C). The decrease in time in ERG amplitudes also correlated with a decrease in cone numbers in this model (FIG. 8D). This was again consistent with the fact that the approach did not stop the degeneration but allowed for enhanced light sensitivity via GIRK2.

### 5. Efficiency of the Mouse GIRK2 in an In Vitro Test

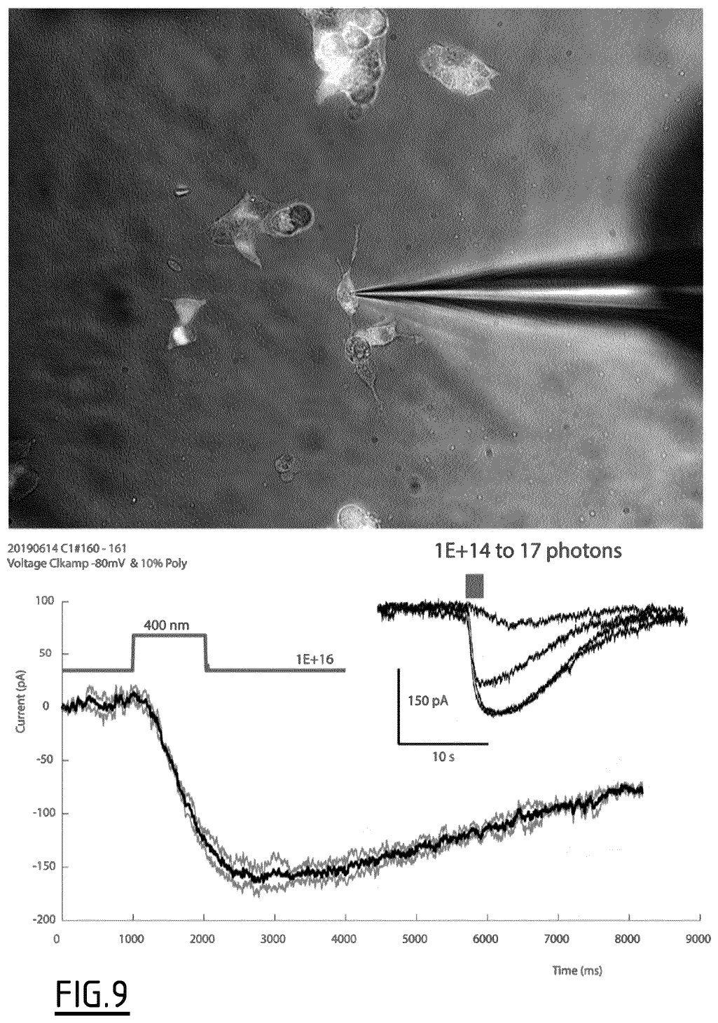

Light stimulations (400 nm, 5 seconds, fullfield) activated GIRK currents in HEK cells expressing both GIRK and SWO (Short Wavelength Opsin) (FIG. 9). GIRK channels are modulated in a membrane-delimited, fast manner via the Gi/o pathway and the expression of the mouse GIRK channel was membrane bound. The amplitudes and kinetics of light-induced activation and deactivation of GIRK channels with SWO, induce large GIRK current amplitudes during a 5 s light pulse.

### Example 3: GIRK1 F137S Elicits Stronger Current than GIRK2 Upon Stimulation with Blue Light (471 nm)

Human embryonic kidney 293 (HEK293) stably expressing mouse Opn4L-mCherry are maintained at 37° C. in Dulbecco's modified Eagle's medium (DMEM), 4.5 g/l D-glucose, supplemented with 10% fetal bovine serum (Gibco) and penicillin/streptomycin in a humidified incubator under 5% CO2. HEK293 cells are transfected with FuGENE® HD (Promega) according to the manufacturer's protocol and incubated for 18-24 h before recordings. Retinaldehyde are added to a final medium concentration of 1 μM.

For GIRK channel recordings GIRK constructs are expressed in HEK293 cells stably expressing Opn4L-mCherry. Cells are cultured and recorded in dark room conditions after transfection. GIRK-mediated K-currents are measured and analyzed as described below. The external solution is as follows: 20 mM NaCl, 120 mM KCl, 2 mM CaCl2, 1 mM MgCl2, 10 mM HEPES-KOH, pH 7.3 (KOH). Patch pipettes (2-5 MΩs) are filled with internal solution: 100 mM potassium aspartate, 40 mM KCl, 5 mM MgATP, 10 mM HEPES-KOH, 5 mM NaCl, 2 mM EGTA, 2 mM MgCl2, 0.01 mM GTP, pH 7.3 (KOH). Cells are recorded in external solution containing 1 μM 9-cis retinal (Sigma). Cells are visualized using a trans-illuminated red light (590 nm) or green light filter (480 nm) during experimental manipulations. Whole-cell patch clamp recordings of HEK293 cells are performed with an EPC10 amplifier (HEKA). Currents are digitized and filtered with the internal 10-KHz three-pole Bessel filter (filter 1) in series with a 2.9-kHz 4-pole Bessel filter (filter 2) of the EPC10 amplifier. Series resistances are partially compensated between 70 and 90%.

For analyzing and comparing GIRK currents HEK293 cells are voltage clamped at −60 mV. For determination of the baseline current a 500 ms long voltage ramp from −100 to +50 mV is applied before light application. Then a 10 sec light pulse of 471 nm is applied at −60 mV. The size of the GIRK currents is related to the conductance of the cell before and after light activation.

For analyzing possible differences in the efficiency of G protein modulation and desensitization between the different GIRK currents we apply light pulses in the range between 0.1-60 sec and vary the intensity of light as described in our publications (Eickelbeck, D. et al. Commun. Biol. 2, (2019); Spoida, K. et al. Melanopsin Curr. Biol. 26, 1206-1212 (2016); Masseck, O. a. O. A. et al. Neuron 81, 1263-1273 (2014)).

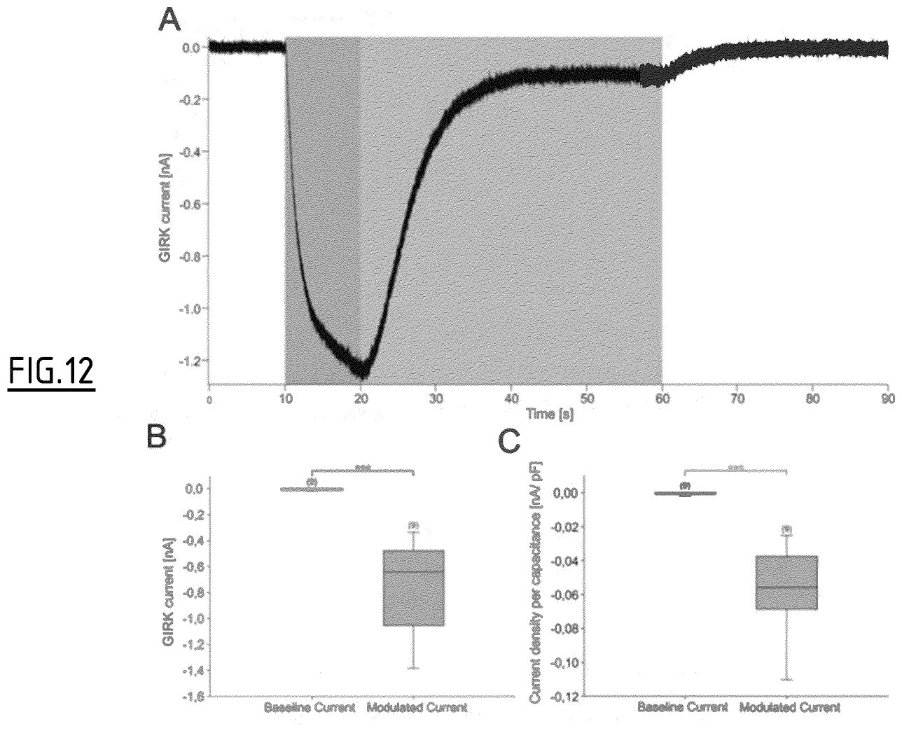

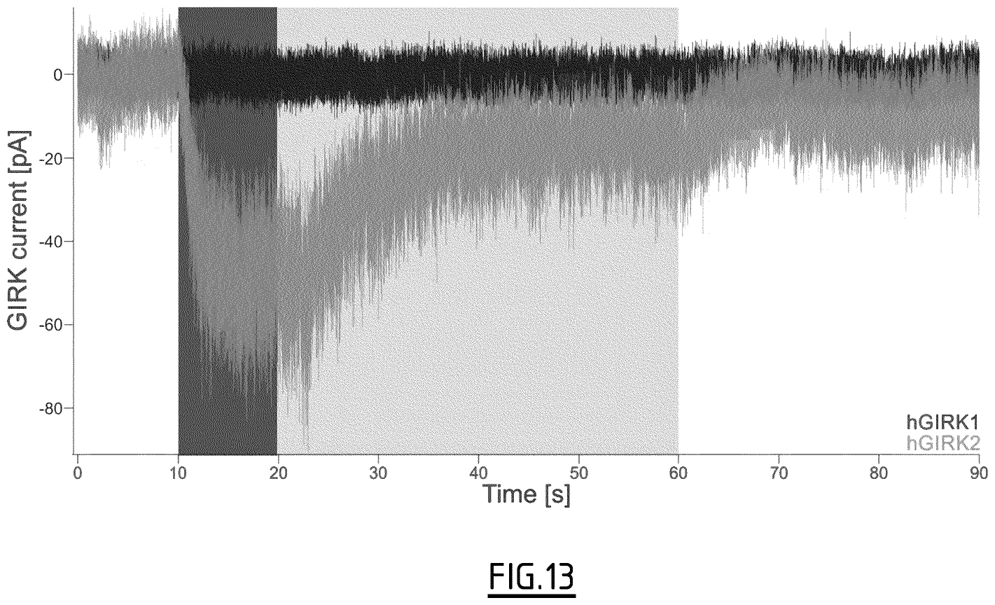

As visible from the comparison of FIGS. 12 and 13, GIRK1 F137S induces significantly more ion efflux than truncated rat GIRK2 (about 17-fold higher) in the context of a short GIRK/opsin phototransduction cascade in HEK cells, while wild-type GIRK1 is ineffective at inducing ion efflux. This result suggests that incorporation of GIRK1 F137S in cones will provide for an improved gene therapy over GIRK2 gene therapy of RCD.

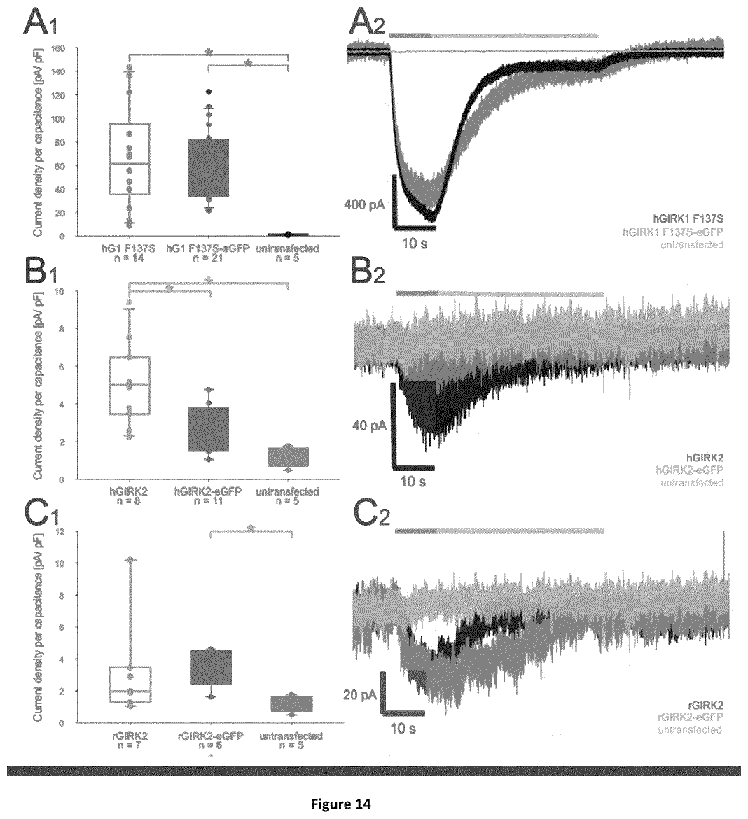

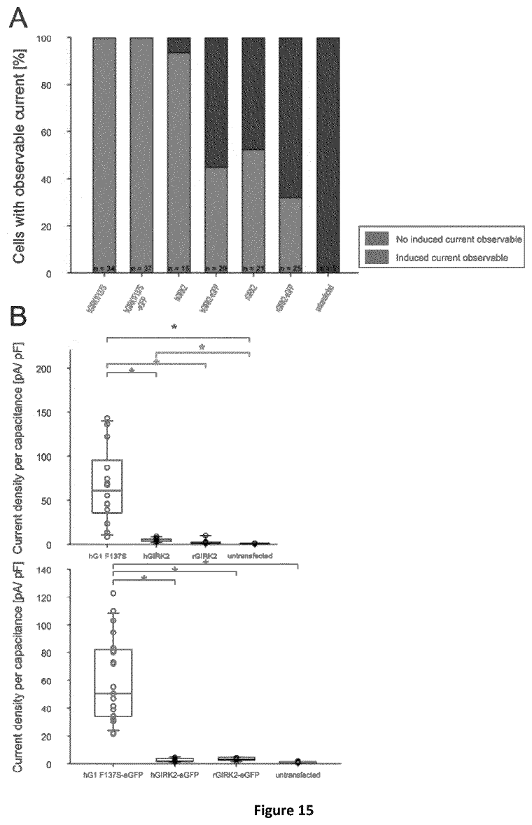

FIGS. 14 and 15 also confirm that GIRK1 F137S (with or without GFP tag) induces significant ion efflux, as compared to either human or rat GIRK2 (with or without GFP tag).

### Example 4: GIRK1 F137S Vision Restoration in an RCD Model Caused by Mutant Rhodopsin

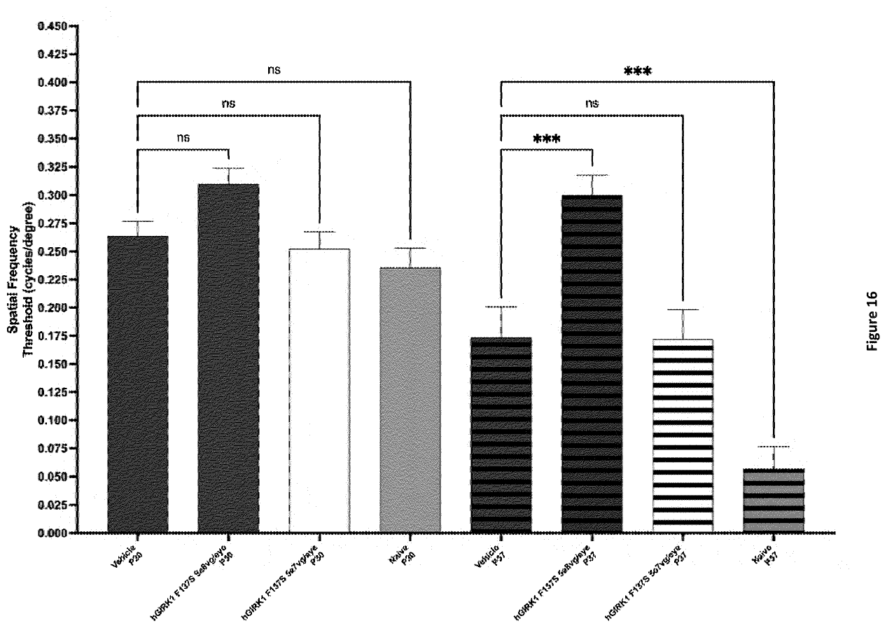

The same experimental model as in Example 2.2 was used in order to demonstrate that AAV-mediated expression of human GIRK1 F137S in the eyes of rd10 mice led to greater visual acuity at P37, as determined by an optokinetic test. The results are shown in FIG. 16. P15 rd10/rd10 mice received a subretinal injection of AAV8-PR1.7-hGIRK1 F137S at a dose of 5e8vg/eye or 5e7vg/eye. At P30 and P37, OKT measurements were performed to assess visual function. Significant visual improvements were observed 3 weeks after administration with 5e8vg/eye, at P37.

### LIST OF REFERENCES

1. Sinha R, Hoon M, Baudin J, Okawa H, Wong R O L, Rieke F. 2017. Cellular and Circuit Mechanisms Shaping the Perceptual Properties of the Primate Fovea. Cell. 168 (3): 413-426.e12
2. Yau K W, Hardie R C. 2009. Phototransduction motifs and variations. Cell. 139 (2): 246-64
3. Ebrey T, Koutalos Y. 2001. Vertebrate photoreceptors. Prog Retin Eye Res. 20 (1): 49-94
4. Larhammar D, Nordström K, Larsson T a. 2009. Evolution of vertebrate rod and cone phototransduction genes. Philosophical transactions of the Royal Society of London. Series B, Biological sciences. 364 (1531): 2867-80
5. Maeda T, Imanishi Y, Palczewski K. 2003. Rhodopsin phosphorylation: 30 Years later. Prog. Retin Eye Res. 22 (4): 417-434
6. Buch H, Vinding T, La Cour M, Appleyard M, Jensen G B, Nielsen N V. 2004. Prevalence and Causes of Visual Impairment and Blindness among 9980 Scandinavian Adults: The Copenhagen City Eye Study. Ophthalmology. 111 (1): 53-61
7. Wright A F, Chakarova C F, Abd El-Aziz M M, Bhattacharya S S. 2010. Photoreceptor degeneration: genetic and mechanistic dissection of a complex trait. Nature reviews. Genetics. 11 (4): 273-84
8. Ferrari S, Di Iorio E, Barbaro V, Ponzin D, Sorrentino F S, Parmeggiani F. 2011. Retinitis pigmentosa: genes and disease mechanisms. Current genomics. 12 (4): 238-49
9. Li Z Y, Kljavin I J, Milam a H. 1995. Rod photoreceptor neurite sprouting in retinitis pigmentosa. The Journal of neuroscience: the official journal of the Society for Neuroscience. 15(8): 5429-38
10. Bennett J. 2017. Taking Stock of Retinal Gene Therapy: Looking Back and Moving Forward. Molecular Therapy. 25 (5): 1076-94
11. Dalkara D, Duebel J, Sahel J-A. 2015. Gene therapy for the eye focus on mutation-independent approaches. Current Opinion in Neurology. 28 (1): 51-60
12. Busskamp V, Picaud S, Sahel J A, Roska B. 2012. Optogenetic therapy for retinitis pigmentosa. Gene Therapy. 19 (2): 169-75
13. Dalkara D, Sahel J-A. 2014. Gene therapy for inherited retinal degenerations. C. R. Biol. 337 (3): 185-192
14. Scholl H P N, Strauss R W, Singh M S, Dalkara D, Roska B, et al. 2016. Emerging therapies for inherited retinal degeneration. Science Translational Medicine. 8 (368): 368rv6-368rv6
15. Baker C K, Flannery J G. 2018. Innovative Optogenetic Strategies for Vision Restoration. Frontiers in Cellular Neuroscience. 12 (September): 1-8
16. Cehajic-Kapetanovic J, Eleftheriou C, Allen A E, Milosavljevic N, Pienaar A, et al. 2015. Restoration of Vision with Ectopic Expression of Human Rod Opsin. Current Biology. 25 (16): 2111-22
17. Gaub B M, Berry M H, Holt A E, Isacoff E Y, Flannery J G. 2015. Optogenetic Vision Restoration Using Rhodopsin for Enhanced Sensitivity. Molecular therapy: the journal of the American Society of Gene Therapy. 23 (10): 1562-71
18. Van Gelder R N, Kaur K. 2015. Vision Science: Can Rhodopsin Cure Blindness. Current Biology. 25 (16): R713-15
19. van Wyk M, Pielecka-Fortuna J, Lowel S, Kleinlogel S. 2015. Restoring the ON Switch in Blind Retinas: Opto-mGluR6, a Next-Generation, Cell-Tailored Optogenetic Tool. PLOS Biology. 13 (5): e1002143
20. Berry M H, Holt A, Levitz J, Visel M, Aghi K, et al. Restoration of high-sensitivity, adapting, patterned vision with a cone opsin. Nature Communications. (2019): 1-12
21. De Silva S R, Barnard A R, Hughes S, Tam S K E, Martin C, et al. 2017. Long-term restoration of visual function in end-stage retinal degeneration using subretinal human melanopsin gene therapy. Proceedings of the National Academy of Sciences. 114 (42): 11211-16
22. Lin B, Koizumi A, Tanaka N, Panda S, Masland R H. 2008. Restoration of visual function in retinal degeneration mice by ectopic expression of melanopsin. Proceedings of the National Academy of Sciences of the United States of America. 105 (41): 16009-14
23. Masseck O A, Spoida K, Dalkara D, Maejima T, Rubelowski J M, et al. 2014. Vertebrate Cone Opsins Enable Sustained and Highly Sensitive Rapid Control of G i/o Signaling in Anxiety Circuitry. Neuron. 81 (6): 1263-73
24. Mark M D, Herlitze S. 2000. G-protein mediated gating of inward-rectifier K+ channels. Eur. J. Biochem. 267 (19): 5830-5836
25. Busskamp V, Duebel J, Balya D, Fradot M, Viney T J, et al. 2010. Genetic Reactivation of Cone Photoreceptors Restores Visual Responses in Retinitis Pigmentosa. Science. 329 (5990): 413-17
26. Packer A M, Roska B, Hausser M. 2013. Targeting neurons and photons for optogenetics. Nature neuroscience. 16 (7): 805-15
27. Khabou H, Garita-Hernandez M, Chaffiol A, Reichman S, Jaillard C, et al. 2018. Noninvasive gene delivery to foveal cones for vision restoration. JCI Insight. 3 (2): 1-18
28. Demande internationale WO 2018134168
29. Byrne L C, Dalkara D, Luna G, Fisher S K, Clerin E, et al. 2015. Viral-mediated RdCVF and RdCVFL expression protects cone and rod photoreceptors in retinal degeneration. Journal of Clinical Investigation. 125 (1): 105-16
30. Millington-Ward S, Chadderton N, O'Reilly M, Palfi A, Goldmann T, et al. 2011. Suppression and Replacement Gene Therapy for Autosomal Dominant Disease in a Murine Model of Dominant Retinitis Pigmentosa. Molecular Therapy. 19 (4): 642-49
31. Kim W. Chan, Jin-Liang Sui, Michel Vivaudou, Diomedes E. Logothetis, 1996, Control of channel activity through a unique amino acid residue of a G protein-gated inwardly rectifying K⁺ channel subunit, Proc Natl Acad Sci USA.; 93 (24): 14193-14198

1. A vector comprising a nucleotide sequence encoding subunit 1 of G-protein-gated inwardly rectifying potassium channel (GIRK1) F137S.

2. The vector according to claim 1 wherein said vector is a virus, chosen from an adeno-associated virus (AAV), an adenovirus, a lentivirus, an SV40 viral vector.

3. A The vector according to claim 1 er 2, wherein the vector is an AAV2 or AAV9 virus comprising a 7 to 11 amino acid long insertion peptide in the GH loop of the VP1 capsid protein, wherein the insertion peptide comprises amino acid sequence LGETTRP (SEQ ID NO: 7).

4. The vector according to claim 1, wherein the nucleotide sequence encoding subunit 1 of G-protein-gated inwardly rectifying potassium channel (GIRK1) F137S is under the control of a cone-specific promoter.

5. The vector according to claim 4, wherein the cone-specific promoter is pR1.7 or a functional variant thereof, or minimal M-opsin promoter, or a GRK promoter or truncated version thereof.

6. The vector according to claim 1, wherein the nucleotide sequence encoding GIRK1 F137S comprises the sequence SEQ ID NO: 3.

7. The vector according to claim 1, wherein the vector is a recombinant AAV9 vector comprising:
a VP1 capsid protein wherein a 7 to 11 amino acid long insertion peptide is inserted in the GH loop of said VP1 capsid protein relative to wild-type AAV9 VP1 capsid protein, at a position localized between amino acids 588 and 589 of wild-type AAV9 VP1 capsid protein, wherein said peptide comprises amino acid sequence LGETTRP (SEQ ID NO: 5); and
the nucleotide sequence encoding GIRK1 F137S under the control of a pR1.7 promoter.

8. The vector according to claim 3, wherein said insertion peptide comprises or consists of amino acid sequence AALGETTRPA (SEQ ID NO: 10), LALGETTRPA (SEQ ID NO: 11), or GLGETTRPA (SEQ ID NO: 12).

9. The vector according to claim 1, further comprising a nucleotide sequence encoding a mammalian cone opsin.

10. A pharmaceutically acceptable carrier comprising the vector defined in claim 1.

11. The pharmaceutically acceptable carrier according to claim 10, which further comprises a vector comprising a nucleotide sequence encoding a mammalian cone opsin.

12. The pharmaceutically acceptable carrier according to claim 11 wherein the vector comprising a nucleotide sequence encoding a mammalian cone opsin:
a) is selected from the group consisting of an adeno-associated virus (AAV), an adenovirus, a lentivirus, and an SV40 viral vector; or
b) is an AAV2 or AAV9 virus comprising a 7 to 11 amino acid long insertion peptide in the GH loop of the VP1 capsid protein, wherein the insertion peptide comprises amino acid sequence LGETTRP (SEQ ID NO: 7); or
c) is a recombinant AAV9 vector comprising:
a VP1 capsid protein wherein a 7 to 11 amino acid long insertion peptide is inserted in the GH loop of said VP1 capsid protein relative to wild-type AAV9 VP1 capsid protein, at a position localized between amino acids 588 and 589 of wild-type AAV9 VP1 capsid protein, wherein said peptide comprises amino acid sequence LGETTRP (SEQ ID NO: 7); and
the nucleotide sequence encoding the mammalian cone opsin under the control of a pR1.7 promoter.

13. The pharmaceutically acceptable carrier according to claim 10, wherein the carrier is chosen from the group consisting of solid-lipid nanoparticles, chitosan nanoparticles, liposomes, lipoplexes and cationic polymers.

14. The vector according claim 1 or a pharmaceutically acceptable carrier comprising the vector, wherein the mammalian cone opsin is a short wavelength cone opsin (SWO).

15. A pharmaceutical composition comprising the vector according to claim 1, or a pharmaceutically acceptable carrier comprising the vector with a pharmaceutically acceptable, diluent or excipient.

16. The pharmaceutical composition according to claim 15, further comprising a vector comprising a nucleotide sequence encoding a mammalian cone opsin.

17. A method of treating rod-cone dystrophy (RCD) in a subject in need thereof, comprising administering to the subject a therapeutically effective amount of the vector of claim 1, or a pharmaceutically acceptable carrier comprising the vector or a pharmaceutical composition comprising the vector.

18. The method according to claim 17, wherein the vector, the pharmaceutically acceptable carrier or the pharmaceutical composition is administered by subretinal injection at a distance from the fovea.

19. The method according to claim 17, wherein the vector, the pharmaceutically acceptable carrier or the pharmaceutical composition is administered by subretinal injection a) in a region adjacent to a superior or inferior temporal branch of a retinal artery; b) at a distance of 2-3 optic disk diameters away from a center of the fovea; and/or c) at a position localized in a geometric shape delineated by branches of a temporal retinal artery and a temporal retinal vein.

20. The method according to claim 17, wherein the vector, the pharmaceutically acceptable carrier or the pharmaceutical composition is administered by intravitreal injection.

21. A method of treating rod-cone dystrophy (RCD) in a subject in need thereof comprising administering to the subject a therapeutically effective amount of a nucleic acid comprising a sequence encoding subunit 1 of G-protein-gated inwardly rectifying potassium channel (GIRK1) F137S.

22. (canceled)

23. (canceled)

24. (canceled)

25. (canceled)

26. (canceled)
